(19) United States
(12) Patent Application Publication
(10) Pub. No.: US 2005/0215614 A1
(43) Pub. Date: Sep. 29, 2005

(54) Substituted indoles and their use as hcv inhibitors

(75) Inventors: Rajinder Singh, Belmont, CA (US); Ihab S. Darwish, San Mateo, CA (US); Rao S.S. Kolluri, Foster City, CA (US); Sambaiah Thota, Foster City, CA (US); Henry H Lu, Foster City, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Rigel Pharmaceuticals, Inc., South San Francisco, CA (US)

(21) Appl. No.: 10/530,767

(22) PCT Filed: Oct. 15, 2003
(86) PCT No.: PCT/US03/32947

§ 371 Date: Apr. 7, 2005

(51) Int. Cl. A61K 31/4439; A61K 31/405; C07D 49/02; C07D 43/02; C07D 209/04

(52) U.S. Cl. 514/414; 514/419; 548/465; 548/509; 514/337; 546/277.4

(57) ABSTRACT

The present invention comprises indole-derivatives that are inhibitors of HCV. Composition comprising the compounds in combination with a pharmaceutically acceptable carrier are also disclosed, as are methods of using the compounds and compositions to inhibit HCV infection of a cell, particular in the form of treating HCV infection in a mammal.

### CROSS REFERENCE TO RELATED APPLICATIONS

This application claims priority from U.S. Provisional Application Ser. No. 60/419,012, filed Oct. 15, 2002.

### BACKGROUND OF THE INVENTION

1. Field of the Invention

The present invention is in the field of small molecule inhibitors of HCV and methods of using them to inhibit HCV.

2. Summary of the Related Art

The hepatitis C virus (HCV) is one of the most important causes of chronic liver disease in the United States. It accounts for about 15 percent of acute viral hepatitis, 60 to 70 percent of chronic hepatitis, and up to 50 percent of cirrhosis, end-stage liver disease, and liver cancer. Almost 4 million Americans, or 1.8 percent of the U.S. population, have antibody to HCV (anti-HCV), indicating ongoing or previous infection with the virus. Hepatitis C causes an estimated 8,000 to 10,000 deaths annually in the United States.

A distinct and major characteristic of hepatitis C is its tendency to cause chronic liver disease. At least 75 percent of patients with acute hepatitis C ultimately develop chronic infection, and most of these patients have accompanying chronic liver disease.

Chronic hepatitis C varies greatly in its course and outcome. At one end of the spectrum are patients who have no signs or symptoms of liver disease and completely normal levels of serum liver enzymes. Liver biopsy usually shows some degree of chronic hepatitis, but the degree of injury is usually mild, and the overall prognosis may be good. At the other end of the spectrum are patients with severe hepatitis C who have symptoms, HCV RNA in serum, and elevated serum liver enzymes, and who ultimately develop cirrhosis and end-stage liver disease. In the middle of the spectrum are many patents who have few or no symptoms, mild to moderate elevations in liver enzymes, and an uncertain prognosis. Researchers estimate that at least 20 percent of patients with chronic hepatitis C develop cirrhosis, a process that takes 10 to 20 years. After 20 to 40 years, a smaller percentage of patients with chronic disease develop liver cancer. The therapy of chronic hepatitis C has evolved steadily since alpha interferon was first approved for use in this disease more than ten years ago. At the present time, the optimal regimen appears to be a 24- or 48-week course of the combination of pegylated alpha interferon and ribavirin.

Two forms of peginterferon have been developed and studied in large clinical trials: peginterferon alfa-2a (Pegasys®: Hoffman La Roche: Nutley, N.J.) and peginterferon alfa-2b (Pegintron®: Schering-Plough Corporation, Kenilworth, N.J.). These two products are roughly equivalent in efficacy and safety, but have different dosing regimens. Peginterferon alfa-2a is given subcutaneously in a dose of 180 mcg per week. Peginterferon alfa-2b is given subcutaneously weekly in doses of 1.5 mcg per kilogram per week (thus in the range of 75 to 150 mcg per week).

Ribavirin is an oral antiviral agent that has activity against a broad range of viruses. By itself, ribavirin has little effect on HCV, but adding it to interferon increases the sustained response rate by two- to three-fold. For these reasons, combination therapy is now recommended for hepatitis C and interferon monotherapy is applied only when there are specific reasons not to use ribavirin.

Ribavirin is an oral medication, given twice a day in 200-mg capsules for a total daily dose of 800 to 1,200 mg based upon body weight and the form of peginterferon. When combined with peginterferon alfa-2b, the recommended dose of ribavirin is 800 mg per day. When combined with peginterferon alfa-2a, the dose of ribavirin is 1,000 mg for patients who weigh less than 75 kilograms (165 pounds) and 1,200 mg for those who weight more than 75 kilograms. In all situations, ribavirin is given in two divided doses daily.

Peginterferon alfa-2a has been approved for use in chronic hepatitis C in the United States (December 2002). Peginterferon alfa-2b is available for general use.

Combination therapy leads to rapid improvements in serum ALT levels and disappearance of detectable HCV RNA in up to 70 percent of patients. However, long-term improvement in hepatitis C occurs only if HCV RNA disappears during therapy and stays undetectable once therapy is stopped. Among patents who become HCV RNA negative during treatment, a proportion relapse when therapy is stopped. The relapse rate is lower in patents treated with combination therapy compared with monotherapy. Thus, a 48-week course of combination therapy using peginterferon and ribavirin yields a sustained response rate of approximately 55 percent. A similar course of peginterferon monotherapy yields a sustained response rate of only 35 percent. A response is considered “sustained” if HCV RNA remains undetectable for six months or more after stopping therapy.

The optimal duration of treatment varies depending on whether interferon monotherapy or combination therapy is used, as well as by HCV genotype. For patients treated with peginterferon monotherapy, a 48-week course is recommended, regardless of genotype. For patients treated with combination therapy, the optimal duration of treatment depends on viral genotype. Patents with genotypes 2 and 3 have a high rate of response to combination treatment (70 to 80 percent), and a 24-week course of combination therapy yields results equivalent to those of a 48-week course. In contrast, patients with genotype 1 have a lower rate of response to combination therapy (40 to 45 percent), and a 48-week course yields a significantly better sustained response rate. Again, because of the variable responses to treatment, testing for HCV genotype is clinically useful when using combination therapy.

In view of the foregoing, there is a desire for alternative, more effective agents for treating HCV infection.

### SUMMARY OF THE INVENTION

The invention provides compounds and methods for treating HCV infection. The invention provides new inhibitors of HCV.

In a first aspect, the invention provides compounds that are useful as inhibitors of HCV.

In a second aspect, the invention provides a composition comprising an inhibitor of HCV according to the invention and a pharmaceutically acceptable carrier, excipient, or diluent.

In a third aspect, the invention provides a method of inhibiting HCV in a cell, comprising contacting a cell in which inhibition of HCV is desired with an inhibitor of HCV of the invention.

The foregoing merely summarizes certain aspects of the invention and is not intended to be limiting in nature. These aspects and other aspects and embodiments are described more fully below.

### DETAILED DESCRIPTION OF THE PREFERRED EMBODIMENTS

In a first embodiment the invention comprises a compound of formula I: 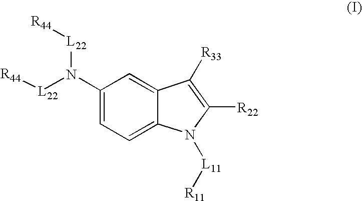
or a pharmaceutically acceptable salt thereof, wherein:
L¹¹ is carboxyl, or a covalent bond when R¹¹ is H;
R¹¹ is H except when L¹¹ is carboxyl, phenyl substituted with 1-3 R⁵⁰, or C₄₋₆-heteroaryl containing 1-3 heteroatoms selected from the group N, S, and O and substituted with 1-3 R⁵⁰;
R²² is H, or C₁₋₆ alkyl, such as CH₃, t-butyl, or neo-pentyl;
R³³ is H, CH₃ or C₁₋₃ alkyl;
each L²² is independently carboxyl (C(O)), C₁₋₄ alkyl C₁₋₄ alkylC(O) or a covalent bond;
each R⁴⁴ is independently H, optionally substituted C₁₋₆ alkyl, optionally substituted C₃₋₇ cycloalkyl, optionally substituted C₃₋₇ heterocycloalkyl containing at least one N, O or S atom, C₃₋₇ cycloalkanone, optionally substituted C₃₋₇ monocyclic or C₇₋₁₃ bicyclic aryl, optionally substituted C₃₋₆ monocyclic or C₅₋₁₃ bicyclic heteroaryl containing at least one N, O, or S atom, or optionally substituted C₃₋₆ monocyclic or C₅₋₁₃ bicyclic heterocycle containing at least one N, O, or S atom, wherein said optional substitutions are one to four R⁶ groups;
each R⁵⁰ is independently H, halo, Cl, F, CF₃, C₁-C₃ per fluoro, C₁-C₃ perhalo, —OC₁-C₃ perhalo, NO₂, CH₃, R⁷, —OCH₃, —OR⁷, —SR⁷, —CN, —NHR⁷, —N(R⁷)₂, —CON(H)R²³CON(R⁷)₂, —R²³N(H)R⁷, —R²³N(R⁷)₂;
each R⁶ is independently H, halo, Cl, F, —CF₃, —NO₂, —R⁵⁰, —SR⁵⁰, —OR⁵⁰, —CN, N(R⁵⁰)₂, —C(O)R⁵⁰, R²³C(O)R⁵⁰, —CON(R⁵⁰)₂, C₄-C₆ cycloalkyl, C₃₋₇ cycloalkanone, C₄₋₆ cycloalkylamine, C₃₋₅ monocyclic or C₅₋₁₃ bicyclic heteroaryl containing at least one N, O, or S atoms or a C₆-C₁₂ monocyclic or bicyclic heterocycle containing at least one N, O, or 5 atom;
R⁷ is H, halo or C₁₋₆ alkyl;
R²³ is a bond or is C₁-C₆ alkyl;
with the proviso that R²² is not CH₃ when R¹¹ is H;

with the further proviso that the compound is not:

| | |
|---|---|
| 278 | 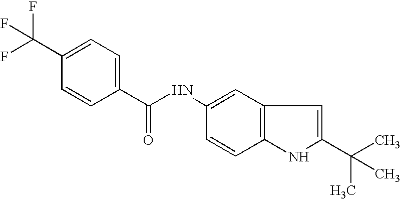 |
| 279 | 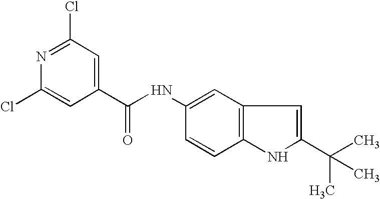 |
| 280 | 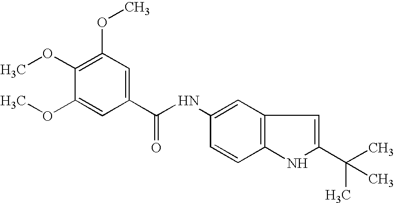 |
| 281 | 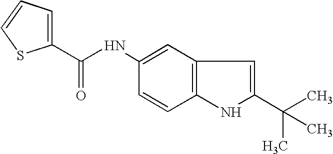 |
| 282 | 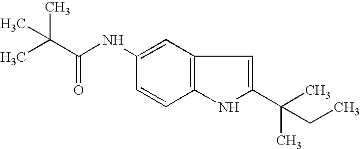 |
| 283 | 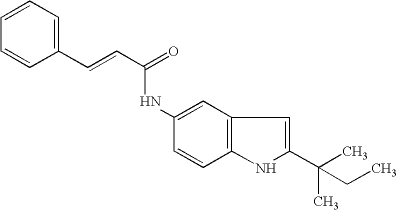 |
| 284 | 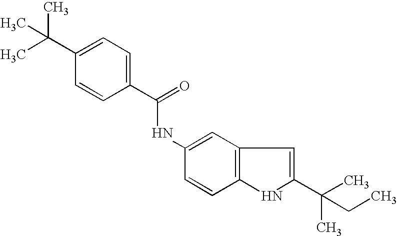 |
| 285 | 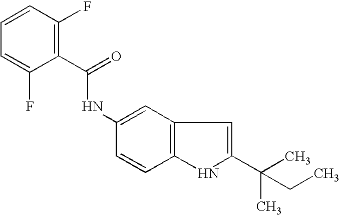 |
| 286 | 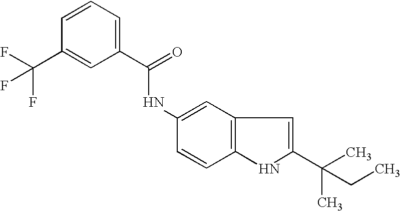 |
| 287 | 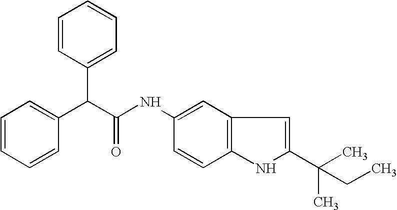 |
| 288 | 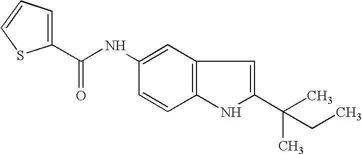 |
| 289 | 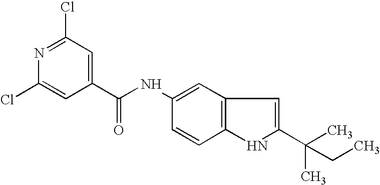 |
| 290 | 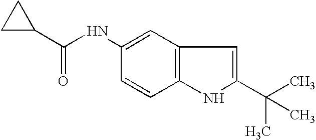 |

In a preferred embodiment of the compounds of paragraph [0018], at least one L²² is carboxyl.

In a preferred embodiment of the compounds of paragraph [0019], the R⁴⁴ attached to said at least one L²² carboxyl is optionally substituted C₁₋₆ alkyl, optionally substituted C₃₋₇ cycloalkyl, optionally substituted C₃₋₇ heterocycloalkyl containing at least one N, O or S atom, C₃₋₇ cycloalkanone, optionally substituted C₅₋₇ monocyclic or C₃₋₁₃ bicyclic aryl, optionally substituted C₃₋₆ monocyclic or C₅₋₁₃ bicyclic heteroaryl containing at least one N, O, or S atom, or optionally substituted C₃-C₁₃ monocyclic or bicyclic heterocycle containing at least one N, O, or S atom.

In a preferred embodiment of the compounds of paragraph [0020], R⁴⁴ is optionally substituted C₃₋₇ cycloalkyl, optionally substituted C₃₋₇ heterocycloalkyl containing at least one N, O or S atom, optionally substituted C₅₋₇ monocyclic aryl, or optionally substituted C₃₋₆ monocyclic heteroaryl containing at least one N, O, or S.

In a preferred embodiment of the compounds of paragraph [0020], R⁴⁴ is optionally substituted C₃₋₇ cycloalkyl, optionally substituted C₅₋₇ monocyclic aryl, or optionally substituted C₃₋₆ monocyclic heteroaryl containing at least one N, O, or S.

In a preferred embodiment of the compounds of paragraph [0018], R²² is t-butyl or Neopentyl.wherein R¹¹ is H.

In a preferred embodiment of the compounds of paragraph [0023], R³³ is H.

In a preferred embodiment of the compounds of paragraph [0022], R²² is t-butyl.

In a preferred embodiment of the compounds of paragraph [0025], R³³ is H.

In a preferred embodiment of the compounds of paragraph [0018], said compound is selected from the group consisting of

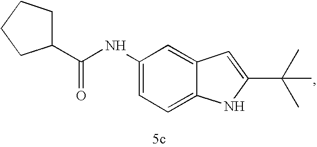

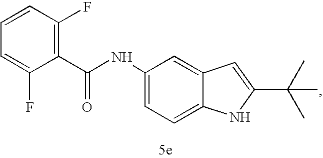

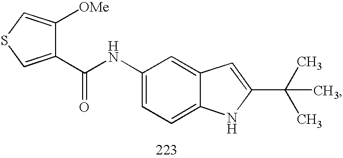

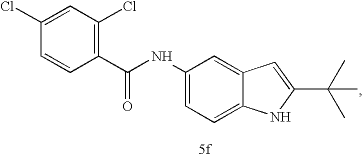

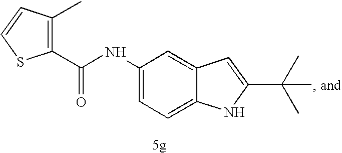

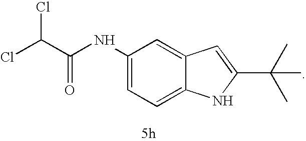

In a preferred embodiment of the compounds of paragraph [0022], R²² is neo-pentyl.

In a preferred embodiment of the compounds of paragraph [0028], R³³ is H.

In a preferred embodiment of the compounds of paragraph [0038], L¹¹ is carboxyl.

In a preferred embodiment of the compounds of paragraph [0030], R¹² is Phenyl or Pyridyl.

In a preferred embodiment of the compounds of paragraph [0020], L¹¹ is carboxyl.

In a preferred embodiment of the compounds of paragraph [0038], R¹¹ is Phenyl or Pyridyl.

In a preferred embodiment of the compounds of paragraph [0033], R²² is CH₃.

In a preferred embodiment of the compounds of paragraph [0034], R³³ is H or CH₃.

In a preferred embodiment of the compounds of paragraph [0033], R¹¹ is substituted with one or two substitutents independently selected from halo, Cl, F, CF₃, CH₃ or —OCH₃.

In a preferred embodiment of the compounds of paragraph [0036], R²² is CH₃.

In a preferred embodiment of the compounds of paragraph [0036], R³³ is H or CH₃.

In a preferred embodiment of the compounds of paragraph [0037], R³³ is H or CH₃.

In a preferred embodiment of the compounds of paragraph [0018], the compound is selected from the group consisting of:

| | |
|---|---|
| 5-(Cyclopentanecarbonyl)amino-1-(2′,6′- dichlorobenzoyl)-3-methylindole, | 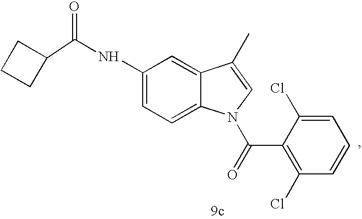 |
| 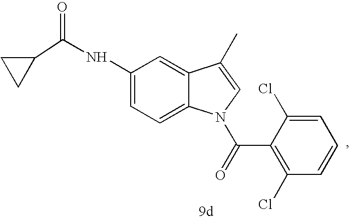 | 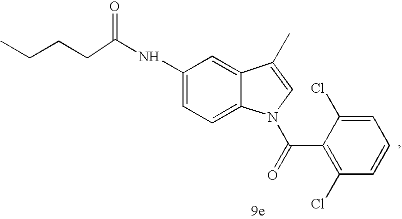 |
| 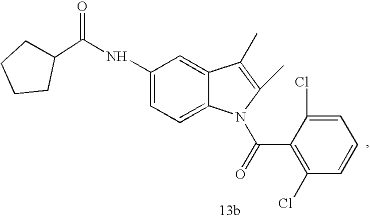 | 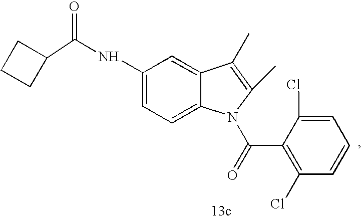 |
| 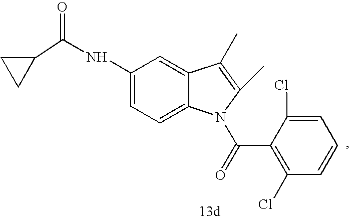 | 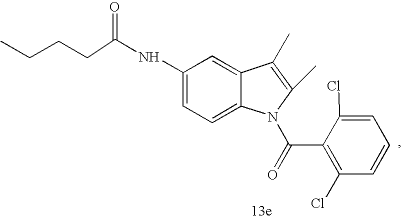 |
| 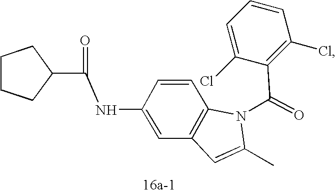 | 1-(2′,6′-Dichlorobenzoyl)-5-(4-methoxy-3- thiophenylcarbonyl)amino-2-methylindole, |
| 1-(2′,6′-Dichlorobenzoyl)-5-(3-pyridyl-2- | 5-Cyclohexanecarbonylamino-1-(2′,6′- |
| acetamido)-2-methylindole, | dichlorobenzoyl)-2-methylindole, |
| 5-Cyclobutanecarbonylamino-1-(2′,6′- | 1-(2′,6′-Dichlorobenzoyl)-5-(3-methyl-2- |
| dichlorobenzoyl)-2-methylindole, | thiophenylcarbonyl)amino-2-methylindole, |
| 1-(2′,6′-Dichlorobenzoyl)-5-(2- | 1-(2′,6′-Dichlorobenzoyl-5-(2- |
| ethylbutanoyl)amino-2-methylindole, | methylpropanoyl)amino-2-methylindole, |
| 1-(2′-Chloro-6′-fluorobenzoyl)-5- | 1-(2′-Chloro-6′-fluorobenzoyl)-5- |
| Cyclohexanecarbonylamino-2-methylindole, | cyclobutanecarbonylamino-2-methylindole, |
| 1-(2′-Chloro-6′-fluorobenzoyl-5- | 1-(2′-Chloro-6′-fluorobenzoyl-5- |
| cyclopropanecarbonylamino-2-methylindole, | cyclopentanecarbonylamino-2-methylindole, |
| 1-(2′-Chloro-6′-fluorobenzoyl)-5-(3-oxo-1- | 1-(2′,6′-Dichlorobenzoyl-5-(2- |
| cyclopentanecarbonyl)amino-2-methylindole, | methylbutanoylamino-2-methylindole, |
| 1-(2′,6′-Dichlorobenzoyl)-5-(n-pentanoyl)amino- | 1-(2′,6′-Dimethoxybenzoyl)-5-(3-oxo-1- |
| 2-methylindole, | cyclopentanecarbonylamino-2-methylindole, |
| 1-(2′,6′-Dichlorobenzoyl)-5-(3-oxo-1- | 1-(2′-Fluoro-6′-trifiuoromethylbenzoyl-5-(3-oxo- |
| cyclopentanecarbonylamino-2-methylindole, | 1-cyclopentanecarbonyl)amino-2- |
| | methylindole, |
| 5-Cyclopropanecarbonylamino-1-(2′,6′- | 5-Cyclopentanecarbonylamino-1-(2′,6′- |
| difluorobenzoyl)-2-methylindole, | difluorobenzoyl)-2-methylindole, |
| 5-Cyclobutanecarbonylamino-1-(2′,6′- difluorobenzoyl)-2-methylindole, | 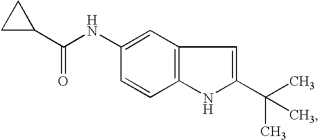 |
| 1-(2′-Chlorobenzoyl)-5- | 1-(o-Anisoyl)-5-cyclopentanecarbonylamino-2- |
| cyclopentanecarbonylamino-2-methylindole, | methylindole, |
| 5-Cyclopentanecarbonylamino-1-(2′,6′- | |
| dichloro-4′-pyridylcarbonyl)-2-methylindole, | |
| 1-(2′,6′-Dichloro-4-pyridylcarbonyl)-5-(3-oxo-1- | 5-Cyclohexanecarbonylamino-1-(2′,6′- |
| cyclopentanecarbonyl)amino-2-methylindole, and | dimethylbenzoyl)-2-methylindole. |

In a preferred embodiment of the compounds of paragraphs [0018] to [0040], at least one L²² is a bond and the R⁴⁴ attached thereto is H.

In a second aspect, the invention comprises a composition comprising a compound of any one of paragraphs [0018]-[0041] and a pharmaceutically acceptable carrier, excipient, or diluent.

In one embodiment of the composition according to paragraph [0042], for said compound at least one L²² is carboxyl.

In a preferred embodiment of the composition according to paragraph [0043], for said compound the R⁴⁴ attached to said at least one L²² carboxyl is optionally substituted C₁₋₆ alkyl, optionally substituted C₃₋₇ cycloalkyl, optionally substituted C₃₋₇ heterocycloalkyl containing at least one N, O or S atom, C₃₋₇ cycloalkanone, optionally substituted C₅₋₇ monocyclic or C₃₋₁₃ bicyclic aryl, optionally substituted C₃₋₆ monocyclic or C₅₋₁₃ bicyclic heteroaryl containing at least one N, O, or S atom, or optionally substituted C₃-C₁₃ monocyclic or bicyclic heterocycle containing at least one N, O, or S atom.

In a preferred embodiment of the composition according to paragraph [0044], for said compound R²² is t-butyl or neo-pentyl.

In a preferred embodiment of the composition according to paragraph [0045], said compound is selected from the group consisting of:

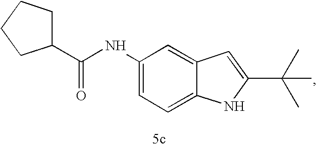

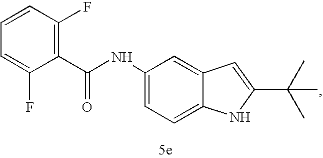

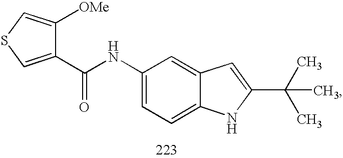

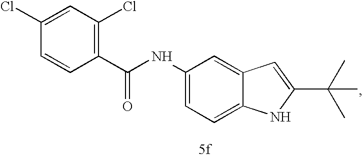

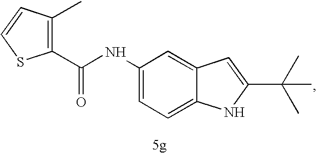

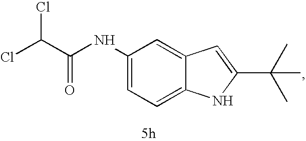

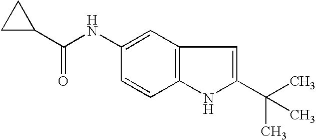

| | |
|---|---|
| 278 | 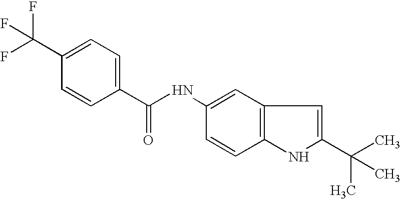 |
| 279 | 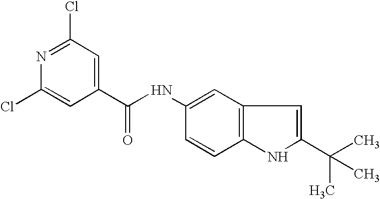 |
| 280 | 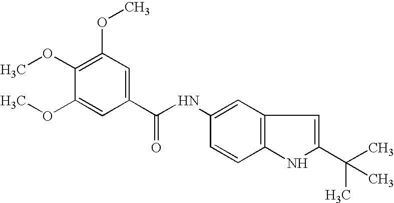 |
| 281 | 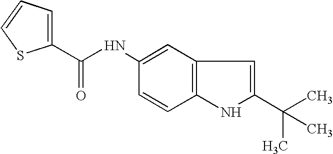 |
| 282 | 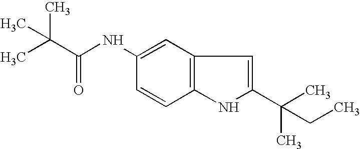 |
| 283 | 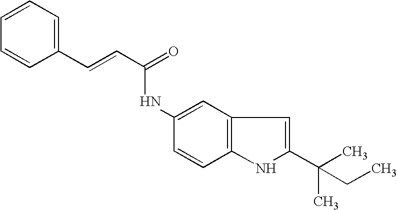 |
| 284 | 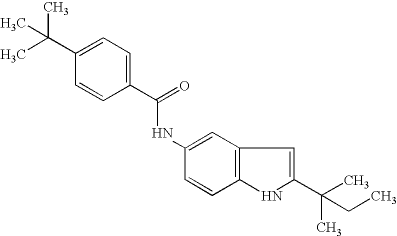 |
| 285 | 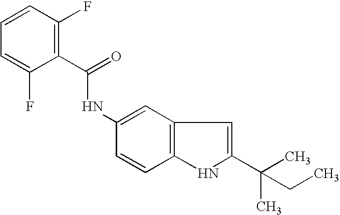 |
| 286 | 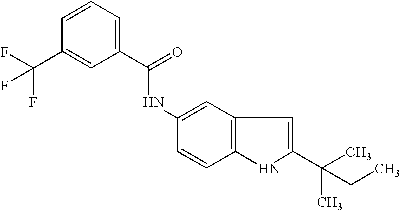 |
| 287 | 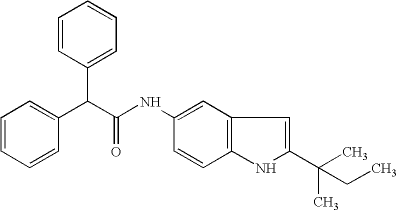 |
| 288 | 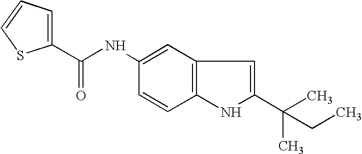 |
| 289 | 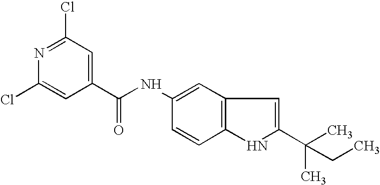 |
| 290 | 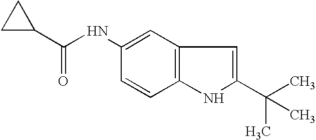 |

In a preferred embodiment of the composition according to paragraph [0044], for said compound L¹¹ is carboxyl.

In a preferred embodiment of the composition according to paragraph [0047], for said compound R¹¹ is Phenyl or Pyridyl.

In a preferred embodiment of the composition according to paragraph [0048], for said compound R²² is CH3.

In a preferred embodiment of the composition according to paragraph [0049], for said compound R³³ is H or CH3.

In a preferred embodiment of the composition according to paragraph [0048], for said compound R³³ is H or CH3.

In a preferred embodiment of the composition according to paragraphs [0042] to [0051], for said compound at least one L²² is a bond and the R⁴⁴ attached thereto is H.

In a third aspect, the invention provides a method of inhibiting HCV in a cell, comprising contacting a cell in which inhibition of HCV is desired with an inhibitor of HCV according to any one of paragraphs [0018]-[0041] or a composition according to paragraph [0042]-0052]. Because compounds of the invention inhibit HCV, they are also useful research tools for in vitro study HCV infections in cells and cellular systems.

In a preferred embodiment of the third aspect, the invention comprises a method of treating an HCV infection in a mammal, preferably a human, comprising administering to the mammal a therapeutically effective amount of a composition according to paragraph [0042]-[0052].

The invention also provides the use of a compound or salt according to formula I for the manufacture of a medicament.

The invention also includes the use of a compound of formula (I) or pharmaceutically acceptable salts thereof for the manufacture of a medicament for use in treating an HCV infection in a mammal.

### DEFINITIONS

Unless expressly stated to the contrary, the following definitions apply uniformly throughout. For simplicity, the substituents have been defined primarily as univalent chemical moieties (e.g., alkyl, aryl, etc.). Nevertheless, such terms are also used to convey corresponding multivalent moieties under the appropriate structural circumstances. All atoms are understood to have their normal number of valences for bond formation (i.e., 4 for carbon, 3 for N, 2 for O, and 2, 4, or 6 for S, depending on the oxidation state of the S). Also, where a chemical structure exists in multiple tautomeric forms, all are envisioned as part of the invention.

The term hydrocarbyl refers to a saturated, mono- or poly-unsaturated straight, branched or cyclic hydrocarbon and specifically includes methyl, ethyl, propyl, isopropyl, butyl, isobutyl, t-butyl, pentyl, cyclopentyl, isopentyl, neopentyl, hexyl, isohexyl, cyclohexyl, 3-methylpentyl, 2,2-dimethylbutyl, 2,3-dimethylbutyl, acetylenyl, propynyl, cyclopropyl, and —C≡C—CH₂(alkyl) (including —C≡C—CH₂(CH₃). A hydrocarbyl moiety may be defined to include a “Cₒ-Cₙ-hydrocarbyl,” “C₀-Cₙ-alkyl,” or the like, in which n is an integer, as in “aryl-C₀-C₃-alkyl.” In these instances a “C₀” moiety represents a direct bond. So, for example, “aryl-C₀-C₃-alkyl” encompasses both aryl-C₁-C₆-alkyl moieties as well as aryl moieties (C₀-alkyl).

An “aryl” group is a C₆-C₁₄ aromatic moiety comprising one to three aromatic rings, which is optionally substituted. Preferably, the aryl group is a C₆-C₁₀ aryl group. Preferred aryl groups include, without limitation, phenyl, naphthyl, anthracenyl, and fluorenyl. An “aralkyl” or “arylalkyl” group comprises an aryl group covalently linked to an alkyl group, either of which may independently be optionally substituted or unsubstituted. Preferably, the aralkyl group is (C₆C₁₀)aryl-(C₁-C₆)alkyl, including, without limitation, benzyl, phenethyl, and naphthylmethyl.

The term heteroatom means O, S, or N.

A “heterocyclyl” group is a mono-, bi-, or tri-cyclic structure having from 3 to 14 atoms, wherein one or more annular atoms are selected from the group consisting of N, O, and S. The heterocyclic group is optionally substituted on carbon at one or more positions. The heterocyclic group is also independently optionally substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocyles include, without limitation, tetrahydroquinoline and dihydrobenzofuran. Specifically excluded from the scope of this term are compounds having adjacent annular O and/or S atoms.

As used herein, the term “heteroaryl” refers to groups having 5 to 14 ring atoms, preferably 5, 6, 9, or 10 ring atoms; having 6, 10, or 14 π electrons shared in a cyclic array; and having, in addition to carbon atoms, from zero to three heteroatoms per ring selected from the group consisting of N, O, and S, provided there is at least one heteroatom. A “heteroaralkyl” or “heteroarylalkyl” group comprises a heteroaryl group covalently linked to an alkyl group, either of which is independently optionally substituted or unsubstituted. Preferred heteroalkyl groups comprise a C₁-C₆ alkyl group and a heteroaryl group having 5, 6, 9, or 10 ring atoms. Specifically excluded from the scope of this term are compounds having adjacent annular O and/or S atoms.

Preferred heterocyclyls and heteroaryls include, but are not limited to, acridinyl, azocinyl, benzimidazolyl, benzofuranyl, benzothiofuranyl, benzothienyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4oxadiazolyl, 1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidiny, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thienyl, triazinyl, 1,2,3-triazolyl, 1,2,4triazolyl, 1,2,5-triazolyl, 1,3,4-triazolyl, and xanthenyl.

Open valences on the radical moieties described herein can occur on any one (or more for divalent radicals) of the atoms within the moiety. For example, the C₃ alkyl moiety includes both propyl and isopropyl. As another example, a divalent C₄ alkylene moiety includes both tetramethylene (—CH₂(CH₂)₂CH₂—) and ethylethylene (—CH(CH₂CH₃)CH₂—).

A moiety that is substituted is one in which one or more hydrogens have been independently replaced with another chemical substituent. As a non-limiting example, substituted phenyls include 2-flurophenyl, 3,4-dichlorophenyl, 3-chloro4-fluoro-phenyl, 2-fluor-3-propylphenyl. As another non-limiting example, substituted n-octyls include 2,4 dimethyl-5-ethyl-octyl and 3-cyclopentyl-octyl. As another example, an oxo-subsituted moiety is one in which both hydrogens of a methylene (—CH₂—) are replaced with an oxygen to form a carbonyl (—CO—).

Substituents can be protected or unprotected as necessary, as known to those skilled in the art or as taught, for example, in Greene, et al., “Protective Groups in Organic Synthesis,” John Wiley and Sons, Third Edition, 1999.

As used herein, the term pharmaceutically acceptable salt(s) refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. In another preferred embodiment, the invention comprises the compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counter-ion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

As employed herein, when a moiety (e.g., cycloalkyl, hydrocarbyl, aryl, heteroaryl, heterocyclic, urea, etc.) is described as “optionally substituted” it is meant that the group optionally has from one to four, preferably from one to three, more preferably one or two, non-hydrogen substituents. Suitable substituents include, without limitation, halo, hydroxy, oxo (e.g., an annular —CH— substituted with oxo is —C(O)—) nitro, halohydrocarbyl, hydrocarbyl, aryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, cyano, oxo, carboxy, formyl, nitro, amino, amidino, guanidino,
(b) C₁-C₅ alkyl or alkenyl or arylalkyl imino, carbamoyl, azido, carboxamido, mercapto, hydroxy, hydroxyalkyl, alkylaryl, arylalkyl, C₁-C₈ alkyl, C₁-C₈ alkenyl, C₁-C₈ alkoxy, C₁-C₈ alkoxycarbonyl, aryloxycarbonyl, C₂-C₈ acyl, C₂-C₈ acylamino, C₁-C₈ alkylthio, arylalkylthio, arylthio, C₁-C₈ alkylsulfinyl, arylalkylsulfinyl, arylsulfinyl, C₁-C₈ alkylsulfonyl, arylalkylsulfonyl, arylsulfonyl, C₀-C₆ Nalkyl carbamoyl, C₂-C₁₅ N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, or any of these rings fused or spiro-fused to a cycloalkyl, heterocyclyl, or aryl, wherein each of the foregoing is further optionally substituted with one more moieties listed in (a), above; and
(c) —(CH₂)ₛ—NR³⁰R³¹, wherein s is from 0 (in which case the nitrogen is directly bonded to the moiety that is substituted) to 6, and R³⁰ and R³¹ are each independently hydrogen, cyano, oxo, carboxamido, amidino, C₁-C₈ hydroxyalkyl, C₁-C₃ alkylaryl, aryl-C₁-C₃ alkyl, C₁-C₈ alkyl, C₁-C₈ alkenyl, C₁-C₈ alkoxy, C₁-C₈ alkoxycarbonyl, aryloxycarbonyl, aryl-C₁-C₃ alkoxycarbonyl, C₂-C₈ acyl, C₁-C₈ alkylsulfonyl, arylalkylsulfonyl, arylsulfonyl, aroyl, aryl, cycloalkyl, heterocyclyl, or heteroaryl, wherein each of the foregoing is further optionally substituted with one more moieties listed in (a), above; or
   R³⁰ and R³¹ taken together with the N to which they are attached form a heterocyclyl or heteroaryl, each of which is optionally substituted with from 1 to 3 substituents from (a), above.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—O—). The nitrogen atom of an acylamino or carbamoyl substituent is additionally substituted. The term “sulfonamido” refers to a sulfonamide substituent attached by either the sulfur or the nitrogen atom. The term “amino” is meant to include NH₂, alkylamino, arylamino, and cyclic amino groups. The term “ureido” as employed herein refers to a substituted or unsubstituted urea moiety.

The term “radical” as used herein means a chemical moiety comprising one or more unpaired electrons.

A moiety that is substituted is one in which one or more hydrogens have been independently replaced with another chemical substituent. As a non-limiting example, substituted phenyls include 2-flurophenyl, 3,4-dichlorophenyl, 3-chloro-4-fluoro-phenyl, 2-fluor-3-propylphenyl. As another non-limiting example, substituted n-octyls include 2,4 dimethyl-5-ethyl-octyl and 3-cyclopentyl-octyl. Included within this definition are methylenes (—CH₂—) substituted with oxygen to form carbonyl —CO—).

An “unsubstituted” moiety as defined above (e.g., unsubstituted cycloalkyl, unsubstituted heteroaryl, etc.) means that moiety as defined above that does not have any of the optional substituents for which the definition of the moiety (above) otherwise provides. Thus, for example, while an “aryl” includes phenyl and phenyl substituted with a halo, “unsubstituted aryl” does not include phenyl substituted with a halo.

Preferred embodiments of a particular genus of compounds of the invention include combinations of preferred embodiments.

As used herein, the term pharmaceutically acceptable salt(s) refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counter-ion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patent treated. The term “therapeutically effective amount” is meant to denote a dosage sufficient to inhibit proliferation of the virus in the patent. A preferred dose of the active compound for all of the above-mentioned conditions is in the range from about 0.01 to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The compound is conveniently administered in any suitable unit dosage form, including but not limited to one containing 1 to 3000 mg, preferably 5 to 500 mg of active ingredient per unit dosage form. An oral dosage of 1-500, preferably 10-250, more preferably 25-250 mg is usually suitable.

The active ingredient should be administered to achieve peak plasma concentrations of the active compound of about 0.001-30 μM, preferably about 0.01-10 μM. This may be achieved, for example, by the intravenous injection of a solution or formulation of the active ingredient, optionally in saline, or an aqueous medium or administered as a bolus of the active ingredient.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterores; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials that modify the physical form of the dosage unit, for example, coatings of sugar, shellac, or enteric agents. See generally “Remington's Pharmaceutical Sciences,” Mack Publishing Co., Easton, Pa.

The active compound or pharmaceutically acceptable salt thereof can be administered as a component of an elixir, suspension, syrup, wafer, chewing gum or the like. Syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as antibiotics, antifungals, other anti-inflammatories, or antiviral compounds.

Solutions or suspensions used for parenteral, intradermal, subcutaneous, or topical application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation (CA) and Gilford Pharmaceuticals (Baltimore, Md.). Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidylcholine, arachadoyl phosphatidylcholine, and cholesterol) in an inorganic solvent that is then evaporated, leaving behind a thin film of dried lipid on the surface of the container. Aqueous solutions of the active compound or its monophosphate, diphosphate, and/or triphosphate derivatives are then introduced into the container. The container is then swirled by hand to free lipid material from the sides of the container and to disperse lipid aggregates, thereby forming the liposomal suspension.

### Synthesis

The compounds of the invention can be synthesized according to the schemes and examples presented below using methods well known to those skilled in the art.

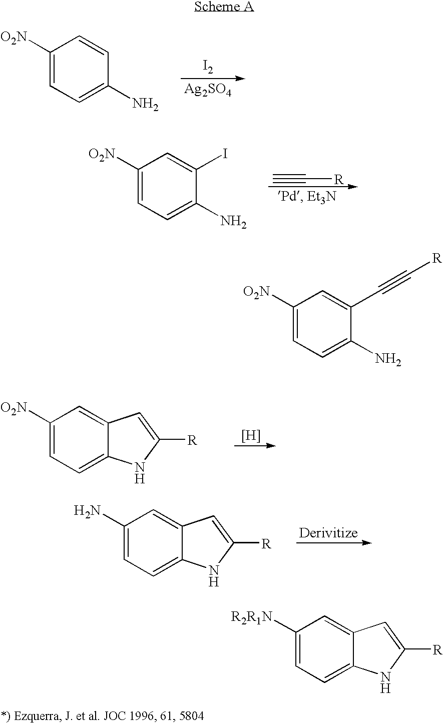

### EXAMPLES

### CHEMISTRY EXAMPLES

Abbreviations and acronyms used in the examples include: AcOH, acetic acid; ACN, acetonitrile; brine, saturated aqueous sodium chloride solution; Cul, copper (I) iodide; d, days; DBU, 1,8-Diazabiyclo[5.4.0]undec-7-ene; DCM, dichloromethane; DEAD, diethylazodicarboxylate; DMF, N,N-dimethylformamide; Et₃N, triethylamine; EtOAc, ethyl acetate; EtOH, ethyl alcohol; h, hour; Fe, iron powder; HPLC, high performance liquid chromatography; LCMS, liquid chromatography mass spectrum (using HPLC); min, minutes; mcpba, 3-chloroperbenzoic acid; MeOH, methanol; MgSO₄, magnesium sulfate; NaHCO₃, sodium bicarbonate; NaOH, sodium chloride; NH₄Cl, ammonium chloride; NMR, nuclear magnetic resonance spectroscopy; ‘Pd’, soluble Palladium catalyst; RP-HPLC, reverse phase-high performance liquid chromatography; rt, room temperature; t-Bu, tertiary-butyl group; TBACl, tetrabutylammonium chloride; THF, tetrahydrofuran; TLC, thin layer chromatography

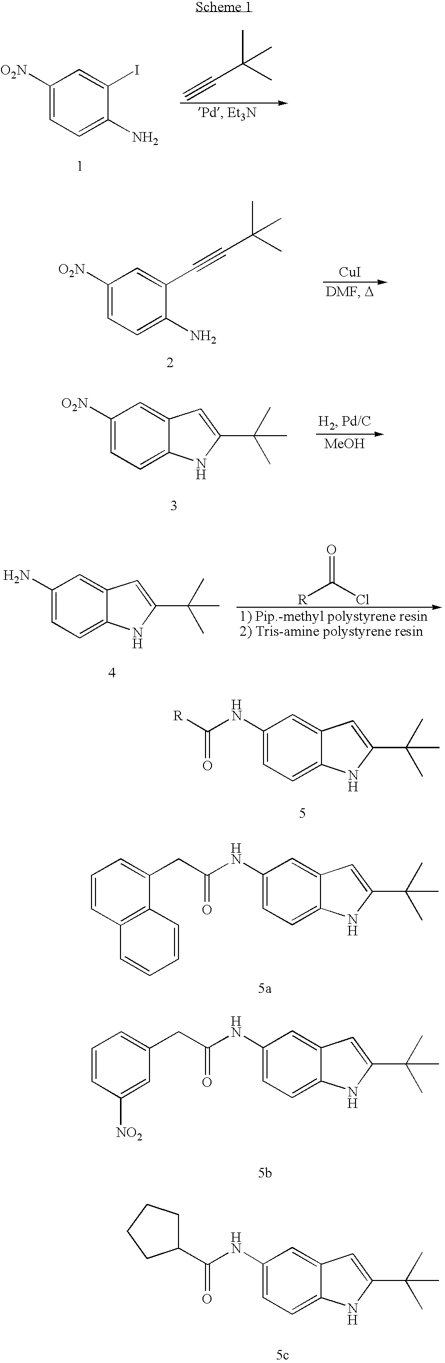

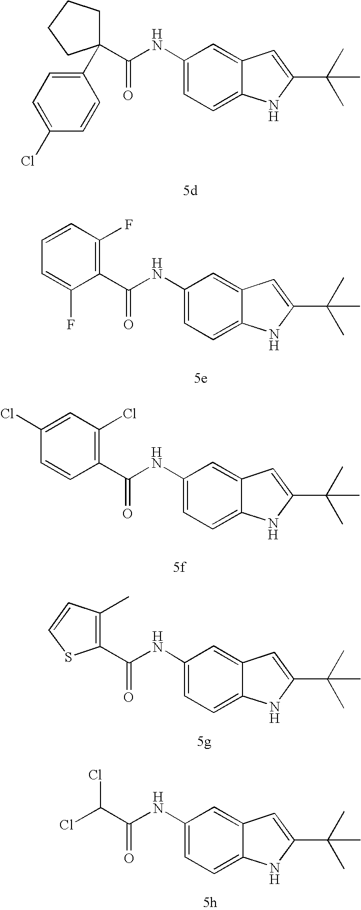

### Synthesis of 2-(3′,3′-Dimethylbut-1-ynyl)-4nitroaniline (2)

In a sealed round-bottom flask, a solution of 2-iodo-4-nitroaniline [Sy, W.-W. Syn. Commun. 1992, 22(22), 3215](1, 5 g, 19 mmol), trans-dichlorobis(triphenyl-phosphine)palladium (II) (665 mg, 0.95 mmol), copper (I) iodide (180 mg, 0.95 mmol)), triethylamine (60 mL) and 3,3-dimethyl-1-butyne (9.3 mL, 6.2 g, 76 mmol) was allowed to stir at room temperature overnight. The reaction mixture was then concentrated in vacuo and the resulting black residue was chromatographed on silica gel (10% ethyl acetate/hexanes). The desired product was isolated and triturated with hexanes to give 2.7 g (65%) as a yellow crystalline solid. ¹H NMR (CDCl₃) [symbol] 8.18 (d, J=2.5 Hz, 1H), 8.00 (dd, J=9.1, 2.5 Hz, 1H), 6.67 (d, J=9.1 Hz, 1H), 1.37 (s, 9H). MS (m/z): 219 (MH⁺).

### Synthesis of 2-t-Butyl-5-nitroindole (3)

A solution of 2-(3′,3-dimethyl-1-butyn-1-yl)-4-nitroaniline (2, 2.7 g, 12.4 mmol), copper (I) iodide (1.2 mg, 6.3 mmol) in DMF (45 mL) was allowed to reflux for 6 h. The volatiles were then removed in vacuo and the resulting black residue was purified on silica gel (20% ethyl acetate/hexanes). The desired product was isolated and triturated with hexanes to give 2.34 g (87%) as a yellow crystalline solid. ¹H NMR (CDCl₃) [symbol] 8.51 (d, J=1.9 Hz, 1H), 8.33 (br s, 1H), 8.07 (dd, J=8.9, 2.3 Hz, 1H), 7.35 (d, J=8.8 Hz, 1H), 6.44 (d, J=1.9 Hz, 1H), 1.44 (s, 9H). MS (m/z): 219 (MH⁺).

### Synthesis of 5-Amino-2-t-butylindole (4)

A solution of 2-t-butyl-5-nitroindole (3, 2.3 g, 11 mmol), Pd/C (10%, 300 mg, 0.3 mmol) in methanol (100 mL) was allowed to mix under 42 psi H₂ atmosphere for 2.25 h. The volatiles were then removed in vacuo and the resulting black residue was purified on silica gel (50% ethyl acetate/hexanes) to give 1.85 g (92%) of the desired product as an orange-brown solid. ¹H NMR (CDCl₃) [symbol] 7.76 (br s, 1H), 7.13 (d, J=8.2 Hz, 1H), 6.88 (d, J=2.2 Hz, 1H), 6.61 (dd, J=8.5, 2.2 Hz, 1H), 6.11 (dd, J=2.3, 1.0 Hz, 1H), 1.39 (s, 9H). MS (m/z): 189 (MH⁺).

### Procedure A. General procedure for the synthesis of 5-Amido-2-t-butylindole analogs (5)

To a solution of 5-amino-2-t-butylindole (4, 0.15 mmol) in DCM (2 mL) was added piperidinomethylpolystyrene resin HL (Novabiochem, 200-400 mesh, 3.7 mmol/g, 0.17 mmol) and acid chloride (0.17 mmol). The resulting reaction mixture was allowed to mix at room temperature overnight after which time Tris-(2-aminoethyl)-aminopolystyrene resin HL (Novabiochem, 200-400 mesh, 3.7 mmol/g, 85 μmol) was added. The resulting reaction mixture was stirred at room temperature for 3.5 h, filtered the solids and washed with DCM. The volatiles were then removed in vacuo and the resulting residue was triturated with an organic solvent.

### 2-t-Butyl-5-(naphth-1-yl-ethanoyl)aminoindole (5a)

Residue triturated with ethyl ether to give a pale yellow solid, wt. 28 mg (55%). ¹H NMR (CD₃OD) [symbol] 10.32 (br s, 1H), 8.17 (d, J=8.2 Hz, 1H), 7.90 (d, J=7.1 Hz, 1H), 7.82 (d, J=8.0 Hz, 1H), 7.60-7.36 (m, 5H), 7.22 (d, J=8.8 Hz, 1H), 7.09 (dd, J=8.5, 1.9 Hz, 1H), 6.09 6.09 (d, J=1.7 Hz, 1H), 4.18 (s, 2H), 1.37 (s, 9H). MS (m/z): 357 (MH⁺).

### 2-t-Butyl-5-(3-nitrophenylethanoyl)aminoindole (5b)

Residue triturated with ethyl ether to give a solid, wt. 36 mg (71%). ¹H NMR (CD₃OD) δ 8.30 (t, J=1.9 Hz, 1H), 8.18-8.14 (m, 1H), 7.80 (d, J=8.0 Hz, 1H), 7.63-7.57 (m, 2H), 7.23 (d, J=8.5 Hz, 1H), 7.10 (dd, J=8.5, 1.9 Hz, 1H), 6.10 (d, J=0.8 Hz, 1H), 3.83 (s, 2H), 1.37 (s, 9H). MS (m/z): 352 (MH⁺).

### 2-t-Butyl-5-(cyclopentanecarbonyl)aminoindole (5c)

Residue triturated with ethyl ether to give a solid, wt. 13 mg (29%). ¹H NMR (CD₃OD) δ 7.61 (d, J=1.9 Hz, 1H), 7.22 (d, J=8.5 Hz, 1H), 7.09 (dd, J=8.7, 2.1 Hz, 1H), 6.10 (s, 1H), 2.86-2.76 (m, 1H), 1.97-1.59 (m, 8H), 1.38 (s, 9H). MS (m/z): 285 (MH⁺).

### 2-t-Butyl-5-(1-(4chlorophenyl)-1-cyclopentanecarbonyl)aminoindole (5d)

Residue triturated with ethyl ether to give a solid, wt. 34 mg (54%). ¹H NMR (CD₃OD) δ 7.48-7.44 (m, 2H), 7.39 (d, J=1.9 Hz, 1H), 7.37-7.33 (m, 2H), 7.19 (d, J=8.8 Hz, 1H), 6.92 (dd, J=8.5, 1.9 Hz, 1H), 6.08 (d, J=0.8 Hz, 1H), 2.67-2.59 (m, 2H), 2.08-1.99 (m, 2H), 1.87-1.76 (m, 4H), 1.36 (s, 9H). MS (m/z): 395 (MH⁺).

### 2-t-Butyl-5-(2′,6′-difluorophenylcarbonyl)aminoindole (5e)

Residue triturated with ethyl ether to give a solid, wt. 33 mg (63%). ¹H NMR (CD₃OD) δ 7.77 (dd, J=1.9, 0.8 Hz, 1H), 7.56-7.46 (m, 1H), 7.30-7.20 (m, 2H), 7.14-7.06 (m, 2H), 6.15 (d, J=0.6 Hz, 1H), 1.40 (s, 9H). MS (m/z): 329 (MH⁺).

### 2-t-Butyl-5-(2′,4′-dichlorophenylcarbonyl)aminoindole (5f)

Residue triturated with hexanes to give a solid, wt. 39 mg (68%). ¹H NMR (CD₃OD) [symbol] 7.76 (dd, J=1.9, 0.6 Hz, 1H), 7.60-7.55 (m, 2H), 7.45 (dd, J=8.2, 1.9 Hz, 1H), 7.29-7.20 (m, 2H), 6.15 (d, J=0.6 Hz, 1H), 1.39 (s, 9H). MS (m/z): 361 (MH⁺).

### 2-t-Butyl-5-(3-methylthiophene-2-carbonyl)aminoindole (5g)

Residue crystallized from ethyl ether to give an off-white solid, wt. 18 mg (36%). ¹H NMR (CD₃OD) [symbol] 7.65 (d, J=1.4 Hz, 1H), 7.48 (d, J=5.0 Hz, 1H), 7.27 (d, J=8.5 Hz, 1H), 7.17 (dd, J=8.5, 1.9 Hz, 1H), 6.98 (d, J=5.0 Hz, 1H), 6.14 (d, J=0.6 Hz, 1H), 2.52 (s, 2H), 1.39 (s, 9H). MS (m/z): 313 (MH⁺).

### 2-t-Butyl-5-dichloroacetamidoindole (5h)

Residue triturated with ethyl ether to give an off-white solid, wt. 17 mg (36%). ¹H NMR (CD₃OD) [symbol] 7.69 (d, J=2.2 Hz, 1H), 7.27 (d, J=8.5 Hz, 1H), 7.15 (dd, J=8.7, 2.1 Hz, 1H), 6.40 (s, 1H), 6.14 (s, 1H), 1.39 (s, 9H). MS (m/z): 299 (MH⁺).

The compounds shown in Table 1 were made in analogous manner to that shown in Scheme 1.

TABLE 1

| Cmpd. | |
|---|---|
| No. | Structure |
| 213 | 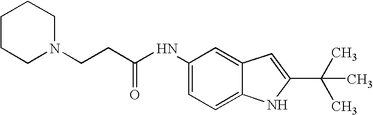 |
| 214 | 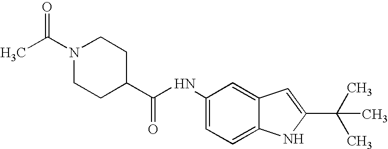 |
| 215 | 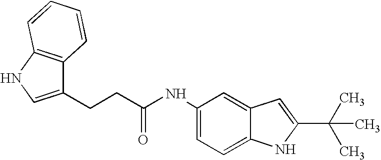 |
| 216 | 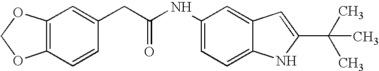 |
| 217 | 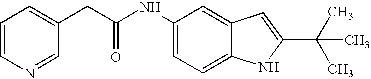 |
| 218 | 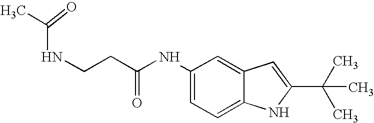 |
| 219 | 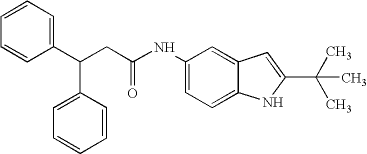 |
| 220 | 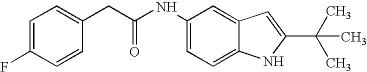 |
| 221 | 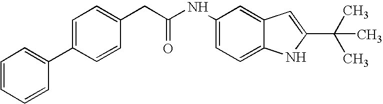 |
| 222 | 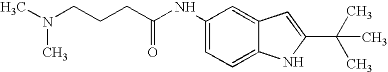 |
| 223 | 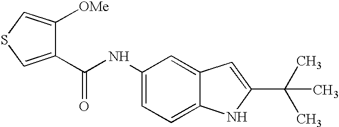 |
| 224 | 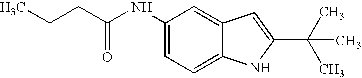 |
| 225 | 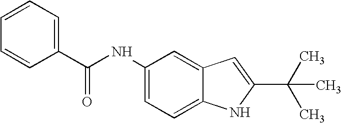 |
| 226 | 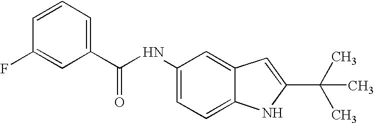 |
| 227 | 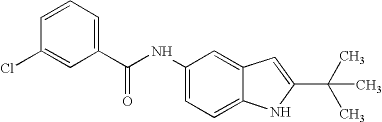 |
| 228 | 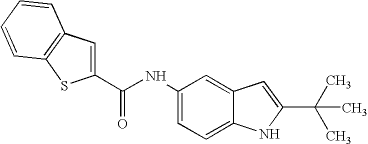 |
| 229 | 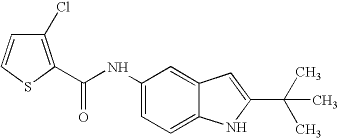 |
| 230 | 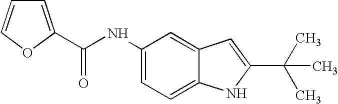 |

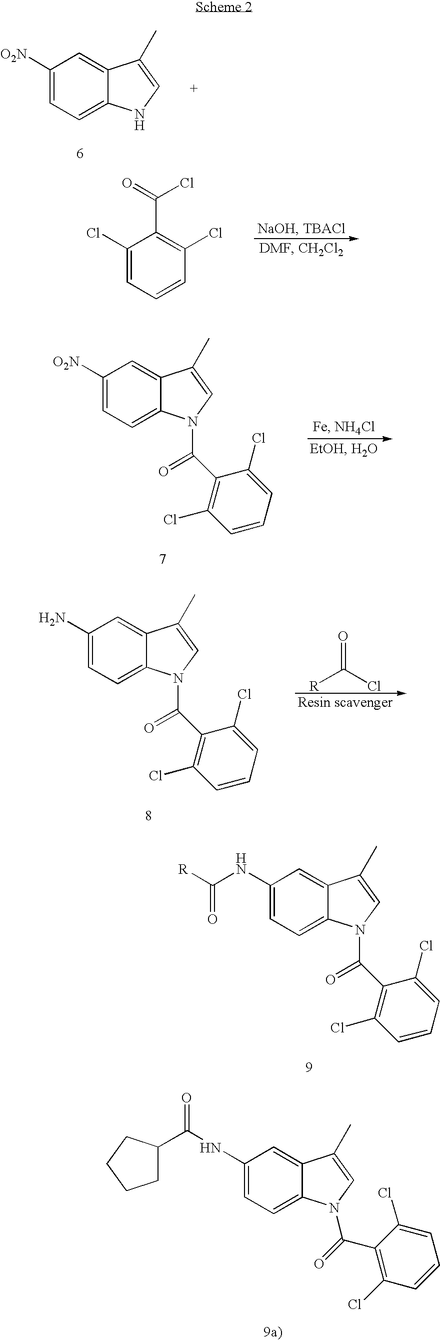

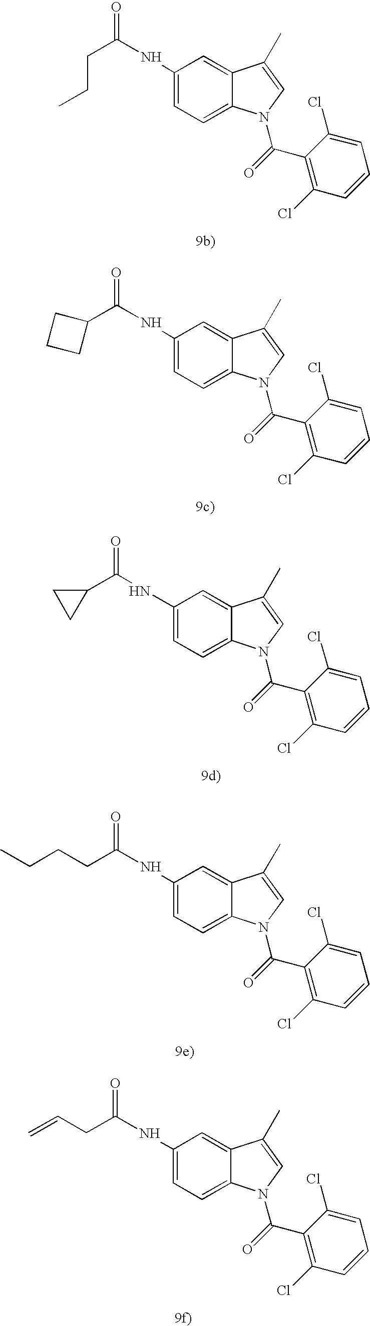

### Synthesis of 1-(2′,6′-Dichlorobenzoyl)-3-methyl-5-nitroindole (7)

To a solution of 3-methyl-5-nitroindole [Katritzky, A. R.; Rachwal, S.; Bayyuk, S. Organic Preparabons and Procedures Int., 1991, 23(3), 357](6, 1.00 g, 5.68 mmol) and tetrabutylammonium chloride (55.6 mg, 0.2 mmol) in methylene chloride (75 mL) and dry DMF (7.5 mL), powdered NaOH (0.290 g, 7.25 mmol) was added under inert atmosphere. 2,6-Dichlorobenzoyl chloride (0.89 mL, 5.68 mmol) in methylene chloride (10 mL) was added to the above reaction mixture over a period of 10 min at ice-cold temperature. After stirring for 1 h at room temperature, the reaction mixture was diluted with water (100 mL), extracted with methylene chloride (3×50 mL), and the organic extracts were dried over Na₂SO₄, filtered and concentrated under reduced pressure. The crude reaction mixture was purified by column chromatography using silica gel to give 1.75 g (88%) of the title compound. ¹H NMR (CDCl₃) [symbol] 8.60 (d, J=9.0 Hz, 1H), 8.40 (d, J=2.3 Hz, 1H), 8.25 (d, J=6.4 Hz, 1H), 7.50 (m, 3H), 6.55 (s, 1H), 2.15 (s, 3H). MS (m/z): 349 (MH⁺).

### Synthesis of 5-Amino-1-(2′,6′-dichlorobenzoyl)-3-methylindole (8)

To a solution of 1-(2′,6′-dichlorobenzoyl)-3-methyl-5-nitroindole (7, 0.5 g, 1.43 mmol) in 2:1 ethanol/water (25 mL), iron powder (0.50 g, 8.9 mmol) was added and allowed the reaction mixture to stir for 10 min, followed by the addition of NH₄Cl (0.162 g, 3.0 mmol). The reaction mixture was stirred for 10 min at room temperature followed by heating to 90° C. for 3 h. The reaction was cooled to room temperature and filtered through a Celite pad. The Celite cake was washed twice with ethanol and the combined filtrate was concentrated in vacuo. The residue was partioned between aqueous NaHCO₃ (100 mL) and methylene chloride (100 mL). The aqueous layer was extracted twice with methylene chloride and the combined organic layer was dried over Na₂SO₄, filtered and concentrated to give the required product in 86% (0.41 g). ¹H NMR (CDCl₃) [symbol] 8.40 (d, J=8.25 Hz, 1H), 7.37-7.42 (m, 3H), 6.77-6.80 (m, 2H), 6.47 (s, 1H), 2.15 (s, 3H). MS (m/z): 319 (MH⁺).

### General procedure for the synthesis of 5-Amido-1-(2′,6′-dichlorobenzoyl)-3-methylindole analogs (9)

As described in procedure A above.

5-(Cyclopentanecarbonyl)amino-1-(2′,6′-dichlorobenzoyl)-3-methylindole (9a) ¹H NMR (CDCl₃) [symbol] 8.50 (d, J=8.53 Hz, 1H), 8.12 (d, J=1.92 Hz, 1H), 7.62 (s, 1H), 7.38-7.42 (m, 3H), 7.23 (m, 1H), 6.52 (s, 1H), 2.70-2.75 (m, 1H), 2.17 (s, 1H), 1.62-1.93 (m, 8H). MS (m/z): 415 (MH⁺).

### 1-(2′,6′-Dichlorobenzoyl)-3-methyl-5-(n-propanecarbonyl)aminoindole (9b)

¹H NMR (CDCl₃) [symbol] 8.52 (d, J=8.53 Hz, 1H), 8.07 (d, J=1.92 Hz, 1H), 7.26-7.41 (m, 4H), 7.19-7.20 (m, 1H), 6.54 (s, 1H), 2.41-2.44 (m, 2H), 2.30 (s, 3H), 1.71-1.76 (m, 2H), 0.91-1.00 (m, 3H). MS (m/z): 389 (MH⁺).

### 5-(Cyclobutanecarbonyl)amino-1-(2′,6′-dichlorobenzoyl)-3-methylindole (9c)

¹H NMR (CDCl₃) [symbol] 8.51 (d, J=8.53 Hz, 1H), 8.11 (d, J=1.92 Hz, 1H), 7.40-7.42 (m, 4H), 7.15-7.20 (m, 1H), 6.54 (s, 1H), 3.20 (m, 1H), 2.44-2.47 (m, 2H), 2.29-2.30 (m, 2H), 2.26 (s, 3H), 2.02-2.20 (m, 2H). MS (m/z): 401 (MH⁺).

### 5-(Cyclopropanecarbonyl)amino-1-(2′,6′-dichlorobenzoyl)-3-methylindole (9d)

¹H NMR (CDCl₃) [symbol] 8.51 (d, J=8.53 Hz, 1H), 8.08 (d, J=1.92 Hz, 1H), 7.60 (s, 1H), 7.40-7.42 (m, 3H), 7.21-7.23 (m, 1H), 6.54 (s, 1H), 2.40 (s, 3H), 1.44 (m, 1H), 0.80-0.77 (m, 2H), 0.70-0.77 (m, 2H). MS (m/z): 387 (MH⁺).

### 5-(n-Butanecarbonyl)amino-1-(2′,6′-dichlorobenzoyl)-3-methylindole (9e)

¹H NMR (CDCl₃) [symbol] 8.50 (d, J=8.53 Hz, 1H), 8.07 (d, J=1.92 Hz, 1H), 7.70 (s, 1H), 7.41 (m, 3H), 7.21-7.24 (m, 1H), 6.53 (s, 1H), 2.31-2.38 (m, 2H), 2.30 (s, 3H), 1.75-1.80 (m, 2H), 1.62-1.70 (m, 2H), 0.91-1.00 (m, 3H). MS (m/z): 403 (MH⁺).

### 5-(trans-Crotonylcarbonyl)amino-1-(2′,6′-dichlorobenzoyl)-3-methylindole (9f)

¹H NMR (CDCl₃) [symbol] 8.56 (d, J=8.53 Hz, 1H), 8.20 (d, J=1.92 Hz, 1H), 7.45 (s, 1H), 7.40-7.45 (m, 3H), 7.20-7.24 (m, 1H), 6.70 (m, 1H), 6.62 (m, 1H), 6.55 (s, 1H), 2.35 (s, 3H), 1.70-1.76 (m, 3H). MS (m/z): 387 (MH⁺).

The compounds shown in Table 2 were made in an analogous manner to that shown in Scheme 2.

TABLE 2

| Com- | |
|---|---|
| pound | Structure |
| 231 | 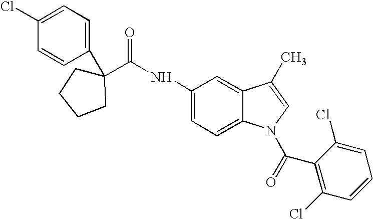 |
| 232 | 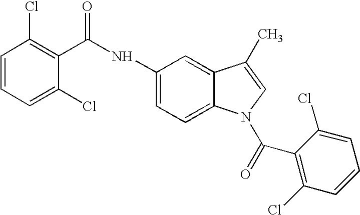 |
| 233 | 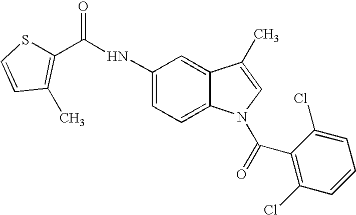 |
| 234 | 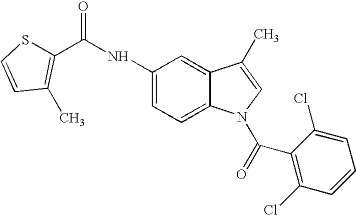 |
| 235 | 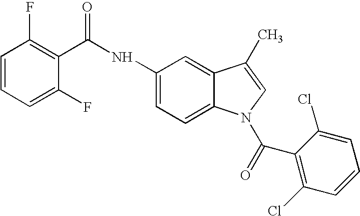 |
| 236 | 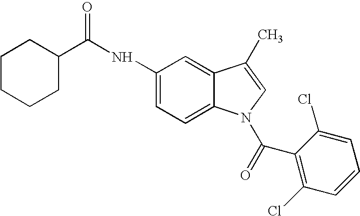 |
| 237 | 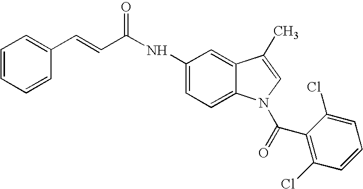 |

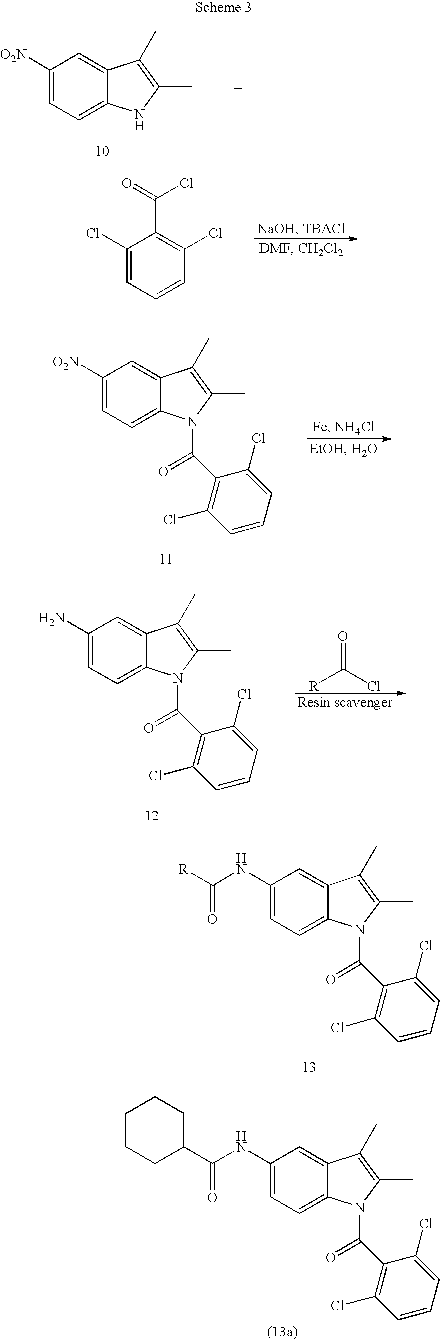

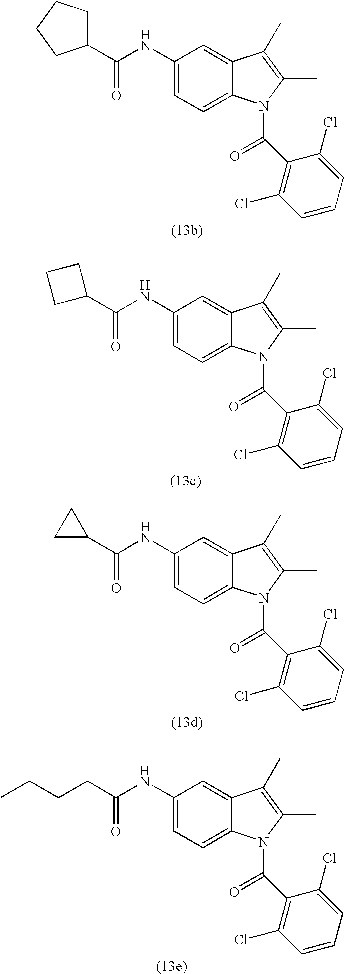

### Synthesis of 1-(2′,6′-dichlorobenzoyl)-2,3-dimethyl-5-nitroindole (11)

To a solution of 2,3-dimethyl-5-nitroindole (10, 1.00 g, 5.26 mmol) and tetrabutylammonium chloride (55.6 mg, 0.2 mmol) in methylene chloride (75 mL) and dry DMF (7.5 mL), powdered NaOH (0.290 g, 7.25 mmol) was added under inert atmosphere. 2,6-dichlorobenzoyl chloride (0.90 mL, 5.3 mmol) in methylene chloride (10 mL) was added to the above reaction mixture over a period of 10 min at ice-cold temperature. After stirring for 1 h at room temperature, the reaction mixture was diluted with water (100 mL), extracted with methylene chloride (3×50 mL), and the organic extracts were combined, dried over Na₂SO₄ and concentrated under reduced pressure. The crude reaction mixture was purified by column chromatography on silica gel to give 1.52 g (77%) of the title compound. ¹H NMR (CDCl₃) [symbol] 8.65 (d, J=9.0 Hz, 1H), 7.55 (m, 1H), 7.30-7.42 (m, 3H), 6.90-6.95 (m, 1H), 2.25 (s, 2H), 2.15 (s, 1H), 2.09 (s, 3H). MS (m/z): 364 (MH⁺).

### Synthesis of 5-Amino-1-(2′,6′-dichlorobenzoyl)-2,3-dimethylindole (12)

To a solution 1-(2′,6′-dichlorobenzoyl-2,3-dimethyl-5-nitroindole (11, 0.5 g, 1.38 mmol) in 2:1 ethanol/water (25 mL), iron powder (0.48 g, 8.7 mmol) was added and stirred for 10 min, followed by the addition of NH₄Cl (0.16 g, 2.88 mmol). The reaction mixture was stirred for 10 min. at room temperature followed by heating to 90° C. for 3 h. The reaction was cooled to room temperature and filtered through Celite pad. The Celite cake was washed twice with ethanol and the combined filtrates were concentrated. The residue was portioned between aqueous NaHCO₃ (100 mL) and methylene chloride (100 mL). The aqueous layer was extracted twice with methylene chloride. The combined organic layers were dried over Na₂SO₄, filtered and concentrated to give the required product in 90% (0.43 g). ¹H NMR (CDCl₃) [symbol] 8.42 (d, J=9.35 Hz, 1H), 7.30-7.42 (m, 3H), 6.70-6.73 (m, 2H), 2.72 (s, 2H), 2.16 (s, 1H), 2.10 (s, 3H). MS (m/z): 334 (MH⁺).

### General procedure for the synthesis of 5-Amino-1-(2′,6′-dichlorobenzoyl)-2,3-dimethylindole analogs (13)

As described in procedure A above.

### 5-(Cyclohexanecarbonyl)amino-1-(2′,6′-dichlorobenzoyl)-2,3-dimethylindole (13a)

¹H NMR (CDCl₃) [symbol] 8.53 (d, J=8.8 Hz, 1H), 8.04 (s, 1H), 7.71 (s, 1H), 7.30-7.42 (m, 3H), 7.11 (m, 1H), 2.74 (s, 2H), 2.20-2.27 (m, 5H), 1.60-1.98 (m, 6H), 1.26-1.37 (m, 4H). MS (m/z): 443 (MH⁺).

### 5-(Cyclopentanecarbonyl)amino-1-(2′,6′-dichlorobenzoyl)-2,3-dimethylindole (13b)

¹H NMR (CDCl₃) [symbol] 8.54 (d, J=8.81 Hz, 1H), 8.05 (s, 1H), 7.30-7.42 (m, 4H), 7.08-7.11 (m, 1H), 2.66-2.74 (m, 3H), 2.21 (s, 1H), 2.19 (s, 3H), 1.89-1.97 (m, 5H), 1.77-1.83 (m, 3H). MS (m/z): 429 (MH⁺).

### 5-(Cyclobutanecarbonyl)amino-1-(2′,6′-dichlorobenzoyl)-2,3-dimethylindole (13c)

¹H NMR (CDCl₃) [symbol] 8.52 (d, J=8.55 Hz, 1H), 8.03 (d, J=1.89 Hz, 1H), 7.43 (s, 1H), 7.34-7.39 (m, 3H), 7.08-7.12 (m, 1H), 2.75-2.80 (m, 3H), 2.45-2.50 (m, 4H), 2.18-2.25 (m, 4H), 2.02-2.20 (m, 2H). MS (m/z): 415 (MH⁺).

### 5-(Cyclopropanecarbonyl)amino-1-(2′,6′-dichlorobenzoyl)-2,3-dimethylindole (13d)

¹H NMR (CDCl₃) [symbol] 8.51 (d, J=8.55 Hz, 1H), 8.00 (s, 1H), 7.60 (s, 1H), 7.30-7.43 (m, 3H), 7.21-7.23 (m, 1H), 2.74 (s, 1H), 2.20 (s, 2H), 2.14 (s, 3H), 1.28 (m, 1H), 1.12-1.14 (m, 2H), 0.91-1.07 (m, 2H). MS (m/z): 401 (MH⁺).

### 5-(n-Butanecarbonyl)amino-1-(2′,6′-dichlorobenzoyl)-2,3-dimethylindole (13e)

¹H NMR (CDCl₃) [symbol] 8.54 (d, J=8.50 Hz, 1H), 8.01 (d, J=1.93 Hz, 1H), 7.30-7.45 (m, 3H), 7.15-7.21 (m, 2H), 2.40-2.45 (m, 4H), 2.30-2.40 (m, 4H), 1.70-1.77 (m, 2H), 1.22-1.30 (m, 2H), 0.91-1.00 (m, 3H). MS (m/z): 417 (MH⁺).

The compounds shown in Table 3 were made in an analogous manner to that shown in Scheme 3.

TABLE 3

| Compound | Structure |
|---|---|
| 238 | 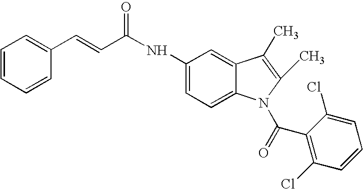 |

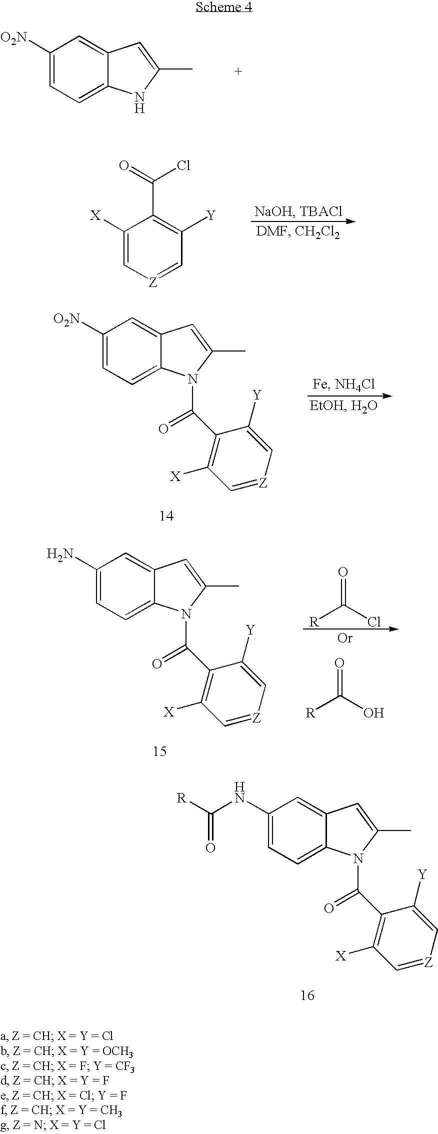

### Procedure B. Representative procedure for the preparation of 1-(2′,6′-Dichlorobenzoyl)-2-methyl-5-nitroindole (14a, Z=CH, X═Y═Cl)

A dry reaction flask equipped with a rubber septum with a N₂ inlet and a magnetic stirring bar was charged with 2-methyl-5-nitroindole (0.5 g, 2.84 mmol), tetrabutylammonium chloride (27.8 mg, 0.1 mmol), powdered NaOH (0.145 g, 3.62 mmol), CH₂Cl₂ (50 mL) and dry DMF (5 mL). A solution of 2,6-dichlorobenzoyl chloride (0.44 ml, 2.84 mmol) in CH₂Cl₂ (10 mL) was added over a period of 10 min to the above reaction at ice-cooled temperature. After stirring for an additional 1 h, the reaction mixture was diluted with ice water (100 mL) and extracted with CH₂Cl₂ (2×50 mL). The organic layers were separated, combined, dried over anhydrous Na₂SO₄ and concentrated in vacuo. The title compound was purified by silica gel column chromatography (30% EtOAc/hexanes) to afford 0.90 g (91%) of the desired product. ¹H NMR (CDCl₃) δ 8.71 (d, J=9.0 Hz, 1H), 8.36 (d, J=2.1 Hz, 1H), 8.24 (d, J=6.3 Hz, 1H), 7.45 (s, 3H), 6.52 (s, 1H), 1.91 (s, 3H). MS (m/z): 349 (MH⁺).

### 1-(2′,6′-Dimethoxybenzoyl)-2-methyl-5-nitroindole (14b, Z=CH, X═Y═OCH₃)

Prepared as described in procedure B above in 80% yield. ¹H NMR (CDCl₃) δ 8.30 (d, J=2.1 Hz, 1H), 8.01 (m, 1H), 7.81 (br s, 1H), 7.43 (t, J=8.4 Hz, 1H), 6.64 (d, J=8.4 Hz, 2H), 6.44 (s, 1H), 3.74 (s, 6H), 2.23 (s, 3H). MS (m/z): 341 (MH⁺).

### 1-(2′-Fluoro-6′-trifluoromethylbenzoyl)-2-methyl-5-nitroindole (14c, Z=CH, X═F, Y═CF₃)

Prepared as described in procedure B above in 96% yield. ¹H NMR (CDCl₃) δ 8.35 (d, J=2.1 Hz, 1H), 8.07 (br s, 1H), 7.60-7.77 (m, 3H), 7.45 (m, 1H), 6.54 (s, 1H), 2.18 (s, 3H). MS (m/z): 367 (MH⁺).

### 1-(2′,6′-Difluorobenzoyl)-2-methyl-5-nitroindole (14d, Z=CH, X═Y═F)

Prepared as described in procedure B above in 85% yield. ¹H NMR (CDCl₃) δ 8.35 (d, J=2.4 Hz, 1H), 8.06 (dd, J=9.3, 2.4 Hz, 1H), 7.74 (d, J=9.0 Hz, 1H), 7.57 (m, 1H), 7.05 (m, 2H), 6.54 (s, 1H), 2.30 (s, 3H). MS (m/z): 317 (MH⁺).

### 1(2′-Chloro-6′-fluorobenzoyl)-2-methyl-5-nitroindole (14e, Z=CH, X═F, Y═Cl)

Prepared as described in procedure B above in 98% yield. ¹H NMR (CDCl₃) δ 8.35 (d, J=2.4 Hz, 1H), 8.09 (dd, J=9.0, 2.1 Hz, 1H), 7.87 (br s, 1H), 7.25 (m, 3H), 6.56 (s, 1H), 2.23 (s, 3H). MS (m/z): 334 (MH⁺).

### 1-(2′,6′-Dimethylbenzoyl)-2-methyl-5-nitroindole (14f, Z=CH, X═Y═CH₃)

Prepared as described in procedure B above in 96% yield. ¹H NMR (CDCl₃) δ 8.25 (d, J=2.4 Hz, 1H), 7.89 (br s, 1H), 7.26 (t, J=7.5 Hz, 1H), 7.04 (d, J=8.4 Hz, 3H), 6.44 (s, 1H), 2.35 (s, 3H), 2.09 (s, 6H). MS (m/z): 309 (MH⁺).

### 1-(2′,6′-Dichloro-4′-pyridylcarbonyl)-2-methyl-5-nitroindole (14g, Z=N, X═Y═Cl)

Prepared as described in procedure B above in 50% yield. ¹H NMR (CDCl₃) δ 8.68 (s, 3H), 8.37 (d, J=2.4 Hz, 1H), 8.24 (br s, 1H), 6.57 (s, 1H), 1.98 (s, 3H). MS (m/z): 391 (MH⁺+41).

### Procedure C. Representative Procedure for the Preparation of 5-Amino-1-(2′,6′-dichlorobenzoyl)-2-methylindole (15a, Z=CH, X═Y═Cl)

A reaction flask equipped with a magnetic stirring bar and a condenser was charged with nitro compound 14a (0.50 g, 1.43 mmol) and 2:1 ethanol/Water (35 mL). Iron powder (0.497 g, 8.88 mmol) was then added and stirred for 10 minutes, followed by the addition of NH₄Cl (0.161 g). The reaction mixture was stirred for 20 minutes at room temperature followed by heating to 80-85° C. for 2 h. The reaction was cooled to room temperature and filtered through a Celite pad. The filter cake was washed with ethanol (2×20 mL) and the filtrate was concentrated in vacuo. The residue was partioned between aq. saturated NaHCO₃ (100 mL) and CH₂Cl₂ (100 mL) and transferred to a separatory funnel. The organic layer was washed with aq. NaHCO₃ (50 mL) and the combined aqueous layers were extracted with CH₂Cl₂ (2×50 mL). The combined organic layers were dried (Na₂SO₄), filtered and the filtrate concentrated to give 0.44 g (95%) of the titled compound. ¹H NMR (CDCl₃) δ 8.43 (d, J=8.7 Hz, 1H), 7.41 (m, 3H), 6.79 (m, 2H), 6.23 (s, 1H), 1.91 (s, 3H). MS (m/z): 319 (MH⁺).

### 5-Amino-1-(2′,6′-dimethoxybenzoyl)-2-methylindole (15b, Z=CH, X═Y═OCH₃)

Prepared as described in procedure C above in 96% yield. ¹H NMR (CDCl₃) δ 7.37 (t, J=8.4 Hz, 1H), 6.70 (d, J=2.4 Hz, 1H), 6.61 (d, J=8.7 Hz, 4H), 6.17 (s, 1H), 3.73 (s, 6H), 1.87 (s, 3H), MS (m/z): 311 (MH⁺).

### 5-Amino-1-(2′-fluoro-6′-trifluoromethylbenzoyl)-2-methylindole (15c, Z=CH, X═F, Y═CF₃)

Prepared as described in procedure C above in 96% yield. ¹H NMR (CDCl₃) δ 8.34 (br s, 1H), 7.61 (br s, 2H), 7.39 (m, 1H), 6.74 (d, J=2.1 Hz, 1H), 6.23 (s, 1H), 2.18 (s, 3H). MS (m/z): 3.37 (MH⁺).

### 5-Amino-1-(2′,6′-difluorobenzoyl)-2-methylindole (15d, Z=CH, X═Y═F)

Prepared as described in procedure C above in 42% yield. ¹H NMR (CDCl₃) δ 7.37 (m, 2H), 6.91 (m, 2H), 6.62 (d, J=2.1 Hz, 1H), 6.44 (dd, J=8.8, 2.1 Hz, 1H), 6.14 (s, 1H), 3.47 (br s, 2H), 2.10 (s, 3H). MS (m/z): 287 (MH⁺).

### 5-Amino-1-(2′-chloro-6′-fluorobenzoyl)-2-methylindole (15e, Z=CH, X═F, Y═Cl)

Prepared as described in procedure C above in 78% yield. ¹H NMR (CDCl₃) δ 7.42 (m, 1H), 7.30 (m, 2H), 7.12 (m, 2H), 6.73 (d, J=2.1 Hz, 1H), 6.25 (s, 1H), 3.28 (br s, 2H), 2.01 (s, 3H). MS (m/z): 303 (MH⁺).

### 5-Amino-1-(2′,6′-dimethylbenzoyl)-2-methylindole (15f, Z=CH, X═Y═CH₃)

Prepared as described in procedure C above in 98% yield. ¹H NMR (CDCl₃) δ 7.18 (m, 1H), 7.01 (d, J=7.2 Hz, 3H), 6.62 (br s, 2H), 6.13 (s, 1H), 2.35 (s, 3H), 2.10 (s, 6H). MS (m/z): 279 (MH⁺).

### 5-Amino-1-(2′,6′-dichloro-4′-pyridylcarbonyl)-2-methylindole (15g, Z=N, X═Y═Cl)

Prepared as described in procedure C above in 98% yield. ¹H NMR (CDCl₃) δ 8.61 (s, 2H), 8.36 (d, J=8.7 Hz, 1H), 6.73 (m, 2H), 6.26 (s, 1H), 3.76 (br s, 2H), 1.98 (s, 3H). MS (m/z): 320 (MH⁺).

### 5-Cyclopentanecarbonylamino-1-(2′,6′-dichlorobenzoyl)-2-methylindole (16a-1, Z=CH, X═Y═Cl)

Prepared as described in procedure A above. ¹H NMR (CDCl) δ 8.52 (d, J=8.7 Hz, 1H), 8.00 (d, J=1.8 Hz, 1H), 7.38 (m, 4H), 7.14 (dd, J=8.7, 2.4 Hz, 1H), 6.32 (s, 1H), 2.78 (m, 1H), 1.94-1.53 (m, 8H), 1.83 (s, 3H). MS (m/z): 415 (MH⁺).

### 5-Cyclohexanecarbonylamino-1-(2′,6′-dichlorobenzoyl)-2-methylindole (16a-2, Z=CH, X═Y═Cl)

Prepared as described in procedure A above. ¹H NMR (CDCl₃) δ 8.50 (d, J=8.7 Hz, 1H), 7.98 (br s, 1H), 7.39 (m, 4H), 7.14 (dd, J=8.7, 1.8 Hz, 1H), 6.31 (s, 1H), 2.26 (m, 1H), 2.03-1.51 (m, 8H), 1.84 (s, 3H), 1.30 (m, 2H). MS (m/z): 429 (MH⁺).

### 5-Cyclobutanecarbonylamino-1-(2′,6′-dichlorobenzoyl)-2-methylindole (16a-3, Z=CH, X═Y═Cl)

Prepared as described in procedure A above. ¹H NMR (CDCl₃) δ 8.51 (d, J=8.7 Hz, 1H), 7.99 (s, 1H), 7.38 (m, 3H), 7.20 (br s, 1H), 7.14 (d, J=9.3 Hz, 1H), 6.33 (s, 1H), 3.17 (m, 1H), 2.41 (m, 2H), 2.51 (m, 2H), 1.96 (m, 2H), 1.84 (s, 3H). MS (m/z): 401 (MH⁺).

### 5-Cyclopropanecarbonylamino-1-(2′,6′-dichlorobenzoyl)-2-methylindole (16a-4, Z=CH, X═Y═Cl)

Prepared as described in procedure A above. ¹H NMR (CDCl₃) δ 8.50 (d, J=8.4 Hz, 1H), 7.95 (s, 1H), 7.81 (br s, 1H), 7.37 (m, 3H), 7.17 (dd, J=9.0, 1.8 Hz, 1H), 6.30 (s, 1H), 3.58 (t, J=5.7 Hz, 1H), 1.83 (s, 3H), 1.57 (m, 2H), 1.09 (m, 1H), 0.82 (m, 1H). MS (m/z): 387 (MH⁺).

### 1-(2′,6′-Dichlorobenzoyl)-5-(2-methylpropanoyl)amino-2-methylindole (16a-5, Z=CH, X═Y═Cl)

Prepared as described in procedure A above. ¹H NMR (CDCl₃) δ 8.52 (d, J=8.7 Hz, 1H), 7.99 (s, 1H), 7.38 (m, 4H), 7.16 (dd, J=8.5, 1.5 Hz, 1H), 6.32 (s, 1H), 2.52 (m, 1H), 1.85 (s, 3H), 1.28 (d, J=6.9 Hz, 6H). MS (m/z): 389 (MH⁺).

### 1-(2′,6′-Dichlorobenzoyl)-5-(2-methylbutanoyl)amino-2-methylindole (16a-6, Z=CH, X═Y═C

Prepared as described in procedure A above. ¹H NMR (CDCl₃) δ 8.51 (d, J=8.7 Hz, 1H), 8.01 (d, J=1.8 Hz, 1H), 7.39 (m, 4H), 7.16 (dd, J=8.7, 2.1 Hz, 1H), 6.32 (s, 1H), 2.28 (m, 1H), 1.84 (s, 3H), 1.78 (m, 1H), 1.54 (m, 1H), 1.26 (d, J=6.6 Hz, 3H), 0.99 (t, J=7.2 Hz, 3H). MS (m/z): 403 (MH⁺).

### 1-(2′,6′-Dichlorobenzoyl)-5-(n-pentanoyl)amino-2-methylindole (16a-7, Z=CH, X═Y═Cl)

Prepared as described in procedure A above. ¹H NMR (CDCl₃) δ 8.51 (d, J=9.0 Hz, 1H), 7.97 (d, J=1.8 Hz, 1H), 7.42 (s, 1H), 7.38 (m, 3H), 7.14 (dd, J=8.7, 2.1 Hz, 1H), 6.33 )s, 1H), 2.39 (d, J=7.5 Hz, 2H), 1.84 (s, 3H), 1.74 (m, 2H), 1.41 (m, 2H), 0.96 (t, J=7.2 Hz, 3H). MS (m/z): 403 (MH⁺).

### 5-(trans-Crotonyl)amino-1-(2′,6′-dichlorobenzoyl)-2-methylindole (16a-8, Z=CH, X═Y═Cl)

Prepared as described in procedure A above. ¹H NMR (CDCl₃) δ 8.44 (d, J=9.3 Hz, 1H), 7.95 (s, 1H), 7.63 (s, 1H), 7.30 (m, 3H), 7.09 (d, J=9.9 Hz, 1H), 6.89 (m, 1H), 6.26 (s, 1H), 5.89 (d, J=14.4 Hz, 1H), 1.85 (d, J=6.9 Hz, 3H), 1.77 (s, 3H). MS (m/z): 387 (MH⁺).

### 1-(2′,6′-Dichlorobenzoyl)-5-(2-ethylbutanoyl)amino-2-methylindole (16a-9, Z=CH, X═Y═Cl)

Prepared as described in procedure A above. ¹H NMR (CDCl₃) δ 8.52 (d, J=8.7 Hz, 1H), 8.02 (d, J=1.8 Hz, 1H), 7.42 (s, 1H), 7.38 (m, 3H), 7.16 (dd, J=8.7, 2.4 Hz, 1H), 6.32 (s, 1H), 2.05 (m, 1H), 1.84 (s, 3H), 1.73 (m, 2H), 1.58 (m, 1H), 0.98 (t, J=7.2 Hz, 3H). MS (m/z): 417 (MH⁺).

### 1-(2′,6′-Dichlorobenzoyl)-5-(3-pyridylcarbonyl)amino-2-methylindole (16a-10, Z=CH, X═Y═Cl)

Prepared as described in procedure A above. ¹H NMR (CDCl₃) δ 9.16 (s, 1H), 8.73 (s, 1H), 8.56 (d, J=6.3 Hz, 1H), 8.41 (d, J=13.8 Hz, 1H), 8.24 (d, J=5.7 Hz, 1H), 8.04 (s, 1H), 7.37 (m, 4H), 7.13 (d, J=8.4 Hz, 1H), 6.36 (s, 1H), 1.86 (s, 3H). MS (m/z): 424 (MH⁺).

### 1-(2′,6′-Dichlorobenzoyl)-5-(3-methyl-2-thiophenylcarbonyl)amino-2-methylindole (16a-11, Z=CH, X═Y═Cl)

Prepared as described in procedure A above. ¹H NMR (CDCl₃) δ 8.55 (d, J=8.7 Hz, 1H), 8.01 (d, J=1.8 Hz, 1H), 7.66 (s, 1H), 7.38 (m, 3H), 7.33 (d, J=4.8 Hz, 1H), 7.02 (dd, J=8.7, 1.8 Hz, 1H), 6.94 (d, J=4.8 Hz, 1H), 6.35 (s, 1H), 2.05 (s, 3H), 1.85 (s, 3H). MS (m/z): 443 (MH³⁰ ).

### 5-Cyclopentanecarbonylamino-1-(2′,6′-dimethoxybenzoyl)-2-methylindole (16b-1, Z=CH, X═Y═OCH₃)

Prepared as described in procedure A above. ¹H NMR (CDCl₃) δ 7.84 (s, 1H), 7.38 (t, J=8.1 Hz, 1H), 7.27 (br s, 1H), 7.02 (d, J=8.1 Hz, 1H), 6.61 (d, J=8.7 Hz, 2H), 6.26 (s, 1H), 3.73 (s, 6H), 2.68 (q, J=8.1 Hz, 1H), 2.15 (s, 3H), 1.93 (m, 4H), 1.79 (m, 2H), 1.62 (m, 2H). MS (m/z): 407 (MH⁺).

### 5-Cyclohexanecarbonylamino-1 (2′,6′-dimethoxybenzoyl)-2-methylindole (16b-2, Z=CH, X═Y═OCH₃)

Prepared as described in procedure A above. ¹H NMR (CDCl₃) δ 7.82 (s, 1H), 7.43 (s, 1H), 7.37 (t, J=8.7 Hz, 1H), 7.02 (d, J=8.1 Hz, 1H), 6.60 (d, J=8.4 Hz, 2H), 6.24 (s, 1H), 3.71 (s, 6H), 2.23 (m, 1H), 1.95 (d, J=12.6 Hz, 2H), 1.82 (s, 3H), 1.69 (m, 2H), 1.55 (m, 2H), 1.27 (m, 4H). MS (m/z): 421 (MH⁺).

### 5-Cyclobutanecarbonylamino-1-(2′,6′-dimethoxybenzoyl)-2-methylindole (16b-3, Z=CH, X═Y═OCH₃)

Prepared as described in procedure A above. ¹H NMR (CDCl₃) δ 7.83 (s, 1H), 7.38 (t, J=8.1 Hz, 1H), 7.24 (s, 1H), 7.03 (d, J=8.4 Hz, 1H), 6.61 (d, J=8.1 Hz, 2H), 6.26 (s, 1H), 3.74 (s, 6H), 3.15 (q, J=8.7 Hz, 1H), 2.40 (m, 3H), 2.21 (s, 3H), 1.95 (m, 3H). MS (m/z): 393 (MH⁺).

### 5-Cyclopropanecarbonylamino-1-(2′,6′-dimethoxybenzoyl)-2-methylindole (16b-4, Z=CH, X═Y═OCH₃)

Prepared as described in procedure A above. ¹H NMR (CDCl₃) δ 7.79 (s, 1H), 7.53 (s, 1H), 7.38 (t, J=8.1 Hz, 1H), 7.03 (d, J=7.5 Hz, 1H), 6.61 (d, J=8.4 Hz, 2H), 6.25 (s, 1H), 3.73 (s, 6H), 3.59 (t, J=5.7 Hz, 1H), 2.13 (s, 3H), 1.08 (m, 2H), 0.81 (m, 2H). MS (m/z): 379 (MH⁺).

### 1-(2′,6′-Dimethoxybenzoyl)-5-(3-pyridylcarbonyl)amino-2-methylindole (16b-5, Z=CH, X═Y═OCH₃)

Prepared as described in procedure A above. ¹H NMR (CDCl₃) δ 9.18 (s, 1H), 8.70 (s, 1H), 8.43 (d, J=10.2 Hz, 1H), 8.22 (d, J=6.3 Hz, 1H), 7.91 (s, 1H), 7.38 (d, J=7.8 Hz, 2H), 7.21 (d, J=8.1 Hz, 1H), 6.60 (d, J=8.1 Hz, 2H), 6.29 (s, 1H), 3.72 (s, 6H), 2.02 (s, 3H), MS (m/z): 406 (MH⁺).

### 5-Cyclopentanecarbonylamino-1-(2′-fluoro-6′-trifluoromethylbenzoyl)-2-methylindole (16c-1, Z=CH, X═F, Y═CF₃)

Prepared as described in procedure A above. ¹H NMR (CDCl₃) δ 8.45 (s, 1H), 7.97 (br s, 2H), 7.39 (m, 2H), 7.11 (br s, 1H), 6.33 (s, 1H), 2.68 (t, J=7.5 Hz, 2H), 1.92 (s, 3H), 1.80 (m, 4H), 1.62 (m, 3H). MS (m/z): 433 (MH⁺).

### 5-Cyclohexanecarbonylamino-1-(2′-fluoro-6′-trifluoromethylbenzoyl)-2-methylindole (16c-2, Z=CH, X═F, Y═CF₃)

Prepared as described in procedure A above. ¹H NMR (CDCl₃) δ 8.45 (s, 1H), 7.96 (s, 1H), 7.62 (br s, 2H), 7.39 (t, J=7.5 Hz, 2H), 7.09 (br s, 1H), 6.33 (s, 1H), 2.24 (t, J=5.7 Hz, 1H), 1.96 (d, J=11.7 Hz, 2H), 1.82 (s, 3H), 1.71 (m, 2H), 1.54 (m, 2H), 1.29 (m, 4H). MS (m/z): 447 (MH⁺).

### 5-Cyclobutanecarbonylamino-1-(2′-fluoro-6′-trifluoromethylbenzoyl)-2-methylindole (16c-3, Z=CH, X═F, Y═CF₃)

Prepared as described in procedure A above. ¹H NMR (CDCl₃) δ 8.37 (s, 1H), 7.88 (s, 1H), 7.54 (br s, 2H), 7.31 (m, 1H), 7.14 (br s, 1H), 7.03 (br s, 1H), 6.25 (s, 1H), 3.07 (t, J=8.1 Hz, 1H), 2.66 (s, 1H), 2.31 (t, J=8.7 Hz, 2H), 2.14 (m, 2H), 1.87 (m, 1H), 1.73 (s, 3H). MS (m/z): 419 (MH⁺).

### 5-Cyclopropanecarbonylamino-1-(2′fluoro-6′-trifluoromethylbenzoyl)-2-methylindole (16c-4, Z=CH, X═F, Y═CF₃)

Prepared as described in procedure A above. ¹H NMR (CDCl₃) δ 8.36 (s, 1H), 7.84 (s, 1H), 7.54 (m, 3H), 7.31 (m, 1H), 7.04 (br s, 1H), 6.24 (s, 1H), 3.51 (t, J=5.7 Hz, 1H), 1.87 (s, 1H), 0.99 (m, 2H), 0.75 (m, 2H). MS (m/z): 405 (MH⁺).

### 5-Cyclopentanecarbonylamino-1-(2′,6′-difluorobenzoyl)-2-methylindole (16d-1, Z=CH, X═Y═F)

Prepared as described in procedure A above. ¹H NMR (CDCl₃) δ 7.86 (d, J=2.1 Hz, 1H), 7.49 (m, 2H), 7.37 (br s, 1H), 7.06 (m, 3H), 6.33 (s, 1H), 2.68 (q, J=8.1 Hz, 1H), 2.24 (s, 3H), 1.93 (m, 3H), 1.80 (m, 3H), 1.62 (m, 2H). MS (m/z): 383 (MH⁺).

### 5-Cyclobutanecarbonylamino-1-(2′,6′-difluorobenzoyl)-2-methylindole (16d-2, Z=CH, X═Y═F)

Prepared as described in procedure A above. ¹H NMR (CDCl₃) δ 7.77 (d, J=1.8 Hz, 1H), 7.41 (m, 2H), 7.09 (br s, 1H), 6.95 (m, 3H), 6.26 (s, 1H), 3.08 (q, J=8.4 Hz, 1H), 2.32 (s, 2H), 2.16 (m, 2H), 2.15 (s, 3H), 1.87 (m, 2H). MS (m/z): 369 (MH⁺).

### 5-Cyclopropanecarbonylamino-1-(2′,6′-difluorobenzoyl)-2-methylindole (16d-3, Z=CH, X═Y═F)

Prepared as described in procedure A above. ¹H NMR (CDCl₃) δ 7.82 (s, 1H), 7.70 (s, 1H), 7.50 (m, 2H), 7.02 (m, 3H), 6.33 (s, 1H), 3.59 (t, J=5.7 Hz, 1H), 2.23 (s, 3H), 1.57 (m, 2H), 1.08 (m, 1H), 0.83 (m, 1H). MS (m/z): 355 (MH⁺).

### 1-(2′-Chloro-6′-fluorobenzoyl)-5-cyclopentanecarbonylamino-2-methylindole (R942617, 16e-1, Z=CH, X═F, Y═Cl)

Prepared as described in procedure A above. ¹H NMR (CDCl₃) δ 7.88 (s, 1H), 7.44 (q, J=6.3 Hz, 2H), 7.37 (br s, 1H), 7.29 (d, J=8.4 Hz, 1H), 7.13 (t, J=8.4 Hz, 1H), 7.13 (t, J=8.4 Hz, 1H), 7.07 (s, 1H), 6.34 (s, 1H), 2.69 (q, J=8.1 Hz, 1H), 2.14 (s, 3H); 1.93 (m, 3H), 1.79 (m, 2H), 1.62 (m, 3H). MS (m/z): 399 (MH⁺).

### 1-(2′-Chloro-6′-fluorobenzoyl)-5-Cyclohexanecarbonylamino-2-methylindole (16e-2, Z=CH, X═F, Y═Cl)

Prepared as described in procedure A above. ¹H NMR (CDCl₃) δ 7.80 (s, 1H), 7.34 (q, J=5.7 Hz, 1H), 7.23 (br s, 1H), 7.20 (d, J=8.4 Hz, 1H), 7.13 (t, J=8.4 Hz, 2H), 7.04 (t, J=8.4 Hz, 1H), 6.98 (s, 1H), 6.26 (s, 1H), 2.15 (m, 1H), 1.90(s, 3H); 1.75 (m, 2H), 1.64 (m, 2H), 1.46 (m, 3H), 1.21 (m, 3H). MS (m/z): 413 (MH⁺).

### 1-(2′-Chloro-6′-fluorobenzoyl)-5-cyclobutanecarbonylamino-2-methylindole (16e-3, Z=CH, X═F, Y═Cl)

Prepared as described in procedure A above. ¹H NMR (CDCl₃) δ 7.88 (s, 1H), 7.44 (m, 1H), 7.28 (d, J=8.1 Hz, 1H), 7.20 (s, 1H), 7.13 (t, J=8.4 Hz, 1H), 7.07 (s, 1H), 6.35 (s, 1H), 3.17 (q, J=8.1 Hz, 1H), 2.41 (s, 3H), 2.21 (m, 3H), 1.99 (m, 3H). MS (m/z): 385 (MH⁺).

### 1-(2′-Chloro-6′-fluorobenzoyl)-5-cyclopropanecarbonylamino-2-methylindole (16e-4, Z=CH, X═F, Y═Cl)

Prepared as described in procedure A above. ¹H NMR (CDCl₃) δ 7.85 (s, 1H), 7.60 (m, 1H), 7.44 (m, 1H), 7.29 (d, J=8.4 Hz, 1H), 7.13 (m, 2H), 7.34 (s, 1H), 3.59 (t, J=5.7 Hz, 1H), 2.15 (s, 3H), 1.09 (m, 2H), 0.85 (m, 2H). MS (m/z): 371 (MH⁺).

### 5-Cyclopentanecarbonylamino-1-(2′,6′-dimethylbenzoyl)-2-methylindole (16f-1, Z=CH, X═Y═CH₃)

Prepared as described in procedure A above. ¹H NMR (CDCl₃) δ 8.50 (s, 1H), 7.85 (s, 1H), 7.35 (s, 1H), 7.28 (t, J=7.8 Hz, 1H), 7.09 (d, J=7.5 Hz, 3H), 6.32 (s, 1H), 2.67 (q, J=7.8 Hz, 1H), 2.17 (s, 6H), 2.10 (s, 3H), 1.91-1.70 (m, 5H), 1.61 (m, 3H). MS (m/z): 375 (MH⁺).

### 5-Cyclohexanecarbonylamino-1-(2′,6′-dimethylbenzoyl)-2-methylindole (16f-2, Z=CH, X═Y═CH₃)

Prepared as described in procedure A above. ¹H NMR (CDCl₃) δ 8.50 (s, 1H), 7.86 (s, 1H), 7.28 (t, J=7.5 Hz, 2H), 7.09 (d, J=7.8 Hz, 2H), 6.32 (s, 1H), 2.22 (s, 3H), 2.17 (s, 6H), 1.95 (m, 2H), 1.82 (m, 2H), 1.69 (m, 2H), 1.55 (m, 3H). MS (m/z): 389 (MH⁺).

### 5-Cyclobutanecarbonylamino-1-(2′,6′-dimethylbenzoyl)-2-methylindole (16f-3, Z=CH, X═Y═CH₃)

Prepared as described in procedure A above. ¹H NMR (CDCl₃) δ 7.86 (s, 1H), 7.28 (t, J=7.8 Hz, 1H), 7.10 (d, J=7.5 Hz, 2H), 7.04 (s, 1H), 6.34 (s, 1H), 3.15 (q, J=8.7 Hz, 1H), 2.40 (m, 2H), 2.22 (m, 2H), 2.18 (s, 6H), 2.02 (s, 3H), 1.97 (m, 2H). MS (m/z): 361 (MH⁺).

### 5-Cyclopropanecarbonylamino-1-(2′,6′-dimethylbenzoyl)-2-methylindole (16f-4, Z=CH, X═Y═CH₃)

Prepared as described in procedure A above. ¹H NMR (CDCl₃) δ 8.47 (s, 1H), 7.75 (s, 1H), 7.28 (t, J=7.8 Hz, 2H), 7.09 (d, J=7.8 Hz, 2H), 6.31 (s, 1H), 3.59 (br s, 1H), 2.17 (s, 6H), 1.07 (m, 2H), 0.81 (m, 2H). MS (m/z): 347 (MH⁺).

### 5-Cyclopentanecarbonylamino-l-(2′,6′-dichloro-4′-pyridylcarbonyl)-2-methylindole (16g-1, Z=N, X═Y═Cl)

Prepared as described in procedure A above. ¹H NMR (CDCl₃) δ 8.66 (s, 1H), 8.62 (s, 1H), 8.47 (d, J=8.4 Hz, 1H), 8.01 (s, 1H), 7.34 (br s, 1H), 7.16 (dd, J=8.8, 2.1 Hz, 1H), 6.36 (s, 1H), 2.69 (q, J=8.1 Hz, 1H), 1.94 (m, 3H), 1.89 (s, 3H), 1.81 (m, 2H), 1.64 (m, 3H). MS (m/z): 416 (MH⁺).

### 5-Cyclopropanecarbonylamino-1-(2′,6′-dichloro-4′-pyridylcarbonyl)-2-methylindole (16g-2, Z=N, X═Y═Cl)

Prepared as described in procedure A above. ¹H NMR (CDCl₃) δ 8.58 (s, 1H), 8.54 (s, 1H), 8.39 (d, J=9.0 Hz, 1H), 7.89 (s, 1H), 7.59 (s, 1H), 7.10 (dd, J=8.6, 2.1 Hz, 1H), 6.27 (s, 1H), 3.51 (br s, 1H), 1.81 (m, 3H), 1.03 (m, 2H), 0.79 (m, 2H). MS (m/z): 388 (MH⁺).

### Procedure D. Representative procedure for the preparation of 1-(2′,6′-Dichlorobenzoyl)-5-(4-methoxy-3-thiophenylcarbonyl)amino-2-methylindole (16a-12. Z=CH, X═Y═Cl)

A dry reaction vial with a cap charged with 5-aminoindole 15a (50 mg, 0.15 mmol), 4-methoxy-3-thiophenecarboxylic acid (35.6 mg, 0.22 mmol) and CH₂Cl₂ (2 mL) was allowed to mix for 10 min. Polystyrenecarbodiimide resin (Argonaut, 1.26 mmol/g, 250 mg, 0.32 mmol) was added and the mixture was allowed to mix for 24 h. Finally tris(2-aminomethyl)-aminopolystyrene resin (Novabiochem, 200-400 mesh, 3.7 mmol/g, 25 mg, 93 μmol) was added to the above vial which was then allowed to stir for 8 h. The reaction mixture was filtered, the resin washed with CH₂Cl₂ (2×3 mL) and the combined organic phases were concentrated and dried in vacuo to give 40.8 mg (57%) of the desired product. ¹H NMR (CDCl₃) δ 9.33 (s, 1H), 8.56 (d, J=8.7 Hz, 1H), 8.22 (d, J=3.6 Hz, 1H), 8.13 (d, J=1.8 Hz, 1H), 7.39 (m, 3H), 7.27 (dd, J=8.5, 2.1 Hz, 1H), 6.42 (d, J=4.2 Hz, 1H), 6.37 (s, 1H), 4.05 (s, 3H), 1.85 (s, 3H). MS (m/z): 459 (MH⁺).

### 1-(2′,6′-Dichlorobenzoyl)-5-(3-pyridyl-2-acetamido)-2-methylindole (16a-13, Z=CH, X═Y═Cl)

Prepared as described in procedure D above. ¹H NMR (CDCl₃) δ 8.83 (s, 1H), 8.56 (s, 1H), 8.51 (d, J=7.8 Hz, 1H), 7.97 (s, 1H), 7.90 (s, 1H), 7.39 (m, 3H), 7.21 (m, 2H), 6.31 (s, 1H), 3.85 (s, 2H), 1.82 (s, 3H). MS (m/z): 438 (MH⁺).

### 1-(2′,6′-Dichlorobenzoyl)-5-(3-oxo-1-cyclopentanecarbonylamino-2-methylindole (16a-14, Z=CH, X═Y═Cl)

Prepared as described in procedure D above. ¹H NMR (CDCl₃) δ 8.45 (d, J=8.4 Hz, 1H), 7.89 (s, 1H), 7.32 (m, 3H), 7.07 (d, J=8.7 Hz, 1H), 6.26 (s, 1H), 3.00 (q, J=6.6 Hz, 1H), 2.60 (d, J=8.7 Hz, 1H), 2.43 (m, 2H), 2.21 (m, 3H), 1.77 (s, 3H). MS (m/z): 429 (MH⁺).

### 1-(2′,6′-Dimethoxybenzoyl)-5-(3-oxo-1-cyclopentanecarbonyl)amino-2-methylindole (16b-6, Z=CH, X═Y═OCH₃)

Prepared as described in procedure D above. ¹H NMR (CDCl₃) δ 7.82 (s, 1H), 7.69 (s, 1H), 7.38 (t, J=8.4 Hz, 1H), 7.03 (d, J=8.1 Hz, 1H), 6.61 (d, J=8.4 Hz, 2H), 6.26 (s, 1H), 3.67 (s, 6H), 3.04 (q, J=7.5 Hz, 1H), 2.68 (d, J=9.0 Hz, 1H), 2.63 (d, J=8.4 Hz, 1H), 2.45 (s, 3H), 2.25 (m, 1H), 2.19 (s, 3H). MS (m/z): 421 (MH⁺).

### 1-(2′-Fluoro-6′-trifluoromethylbenzoyl)-5-(3-oxo-1-cyclopentanecarbonyl)amino-2-methylindole (16c-5, Z=CH, X═F, Y═CF₃)

Prepared as described in procedure D above. ¹H NMR (CDCl₃) δ 8.37 (s, 1H), 7.83 (s, 1H), 7.55 (s, 2H), 7.32 (t, J=8.1 Hz, 2H), 7.01 (s, 1H), 6.27 (s, 1H), 2.97 (br s, 1H), 2.57 (d, J=7.5 Hz, 1H), 2.43 (m, 3H), 2.21 (m, 2H), 2.10 (s, 3H). MS (m/z): 447 (MH⁺).

### 1-(2′-Chloro-6′-fluorobenzoyl)-5-(3-oxo-1-cyclopentanecarbonyl)amino-2-methylindole (16e-5, Z=CH, X═Cl, Y═F)

Prepared as described in procedure D above. ¹H NMR (CDCl₃) δ 7.87 (s, 1H), 7.44 (m, 2H), 7.30 (d, J=8.1 Hz, 1H), 7.14 (t, J=8.4 Hz, 1H), 7.08 (s, 1H), 6.36 (s, 2H), 3.06 (q, J=7.8 Hz, 1H), 2.72 (d, J=8.7 Hz, 1H), 2.66 (d, J=8.4 Hz, 1H), 2.51 (m, 2H), 2.27 (m, 2H), 2.18 (s, 3H). MS (m/z): 413 (MH⁺).

### 1-(2′,6′-Dimethylbenzoyl)-5-(3-oxo-1-cyclopentanecarbonyl)amino-2-methylindole (16f-5, Z=CH, X═Y═CH₃)

Prepared as described in procedure D above. ¹H NMR (CDCl₃) δ 7.82 (s, 1H), 7.35 (s, 1H), 7.29 (t, J=7.8 Hz, 1H), 7.10 (d, J=7.5 Hz, 3H), 6.34 (s, 1H), 3.04 (q, J=7.2 Hz, 1H), 2.65 (d, J=9.9 Hz, 2H), 2.49 (m, 2H), 2.27 (m, 2H), 2.18 (s, 6H), 1.98 (s, 3H). MS (m/z): 389 (MH⁺).

### 1-(2′,6′-Dichloro-4-pyridylcarbonyl)-5-(3-oxo-1-cyclopentanecarbonyl)amino-2-methylindole (16g-3, Z=N, X═Y═Cl)

Prepared as described in procedure D above. ¹H NMR (CDCl₃) δ 8.66 (s, 1H), 8.64 (s, 1H), 8.49 (d, J=9.0 Hz, 1H), 7.99 (d, J=2.1 Hz, 1H), 7.55 (s, 1H), 7.18 (dd, J=8.5, 1.8 Hz, 1H), 6.38 (s, 1H), 3.09 (q, J=7.5 Hz, 1H), 2.67 (m, 2H), 2.51 (m, 2H), 2.29 (m, 2H), 1.90 (s, 3H). MS (m/z): 430 (MH⁺).

The compounds shown in Table 4 were made in an analogous manner to that shown in Scheme 4.

TABLE 4

| Com- | |
|---|---|
| pound | Structure |
| 239 | 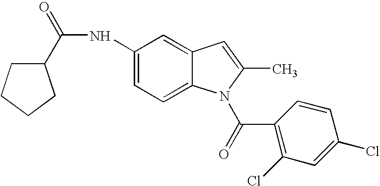 |
| 240 | 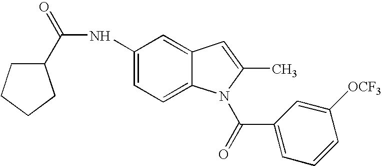 |
| 241 | 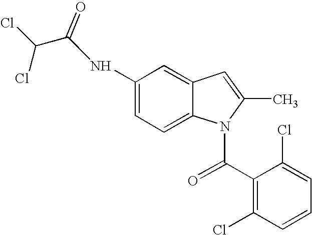 |
| 242 | 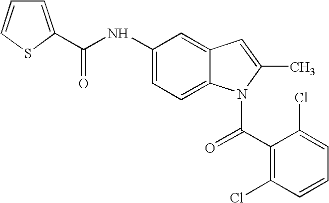 |
| 243 | 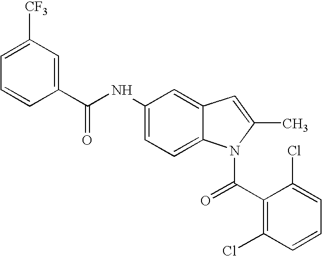 |
| 244 | 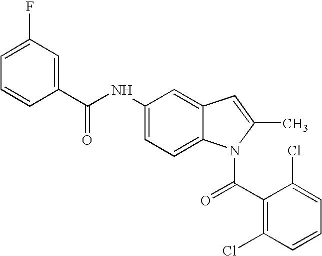 |
| 245 | 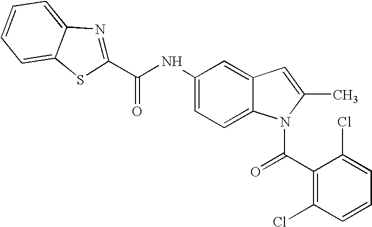 |
| 246 | 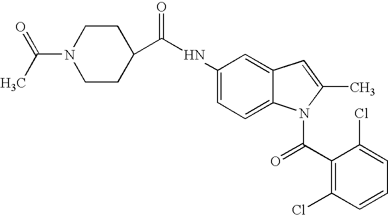 |
| 247 | 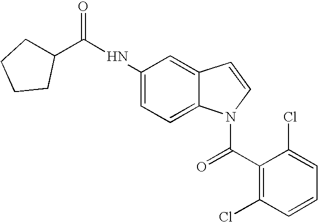 |
| 248 | 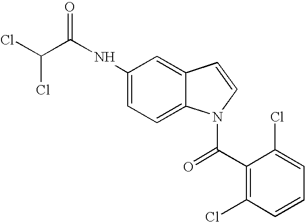 |
| 249 | 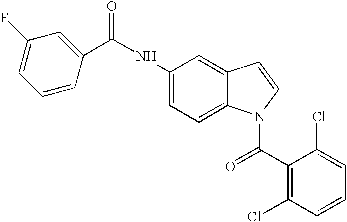 |
| 250 | 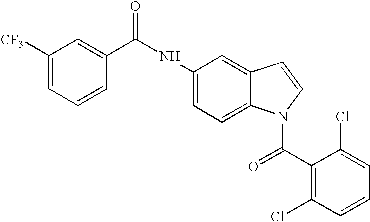 |
| 251 | 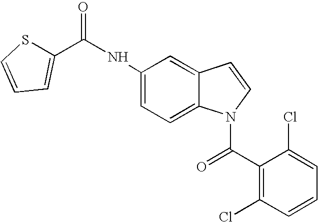 |
| 252 | 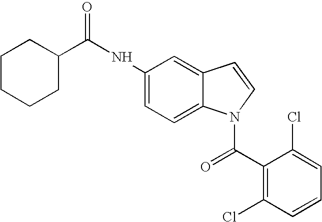 |
| 253 | 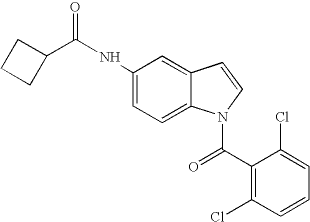 |
| 254 | 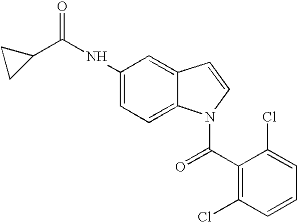 |
| 255 | 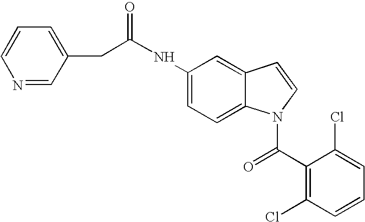 |
| 256 | 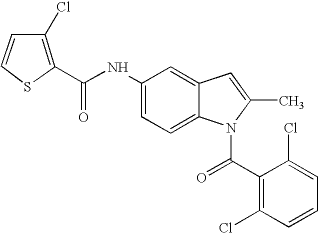 |
| 257 | 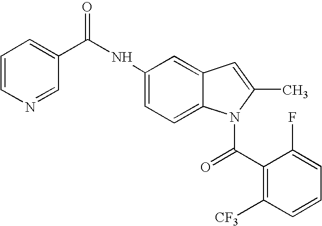 |
| 258 | 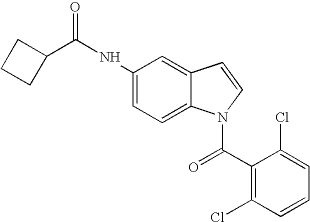 |
| 259 | 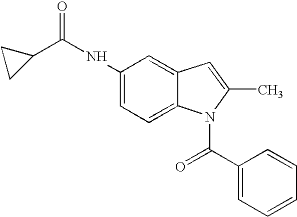 |
| 260 | 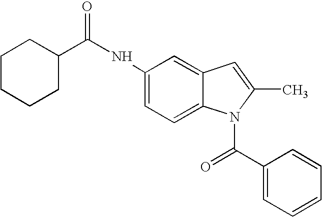 |
| 261 | 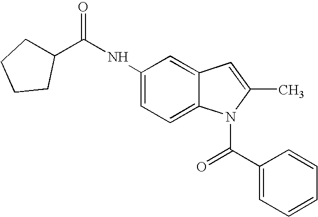 |
| 262 | 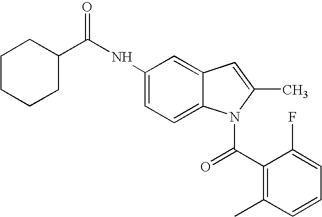 |
| 263 | 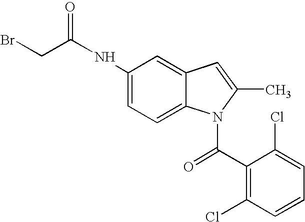 |
| 264 | 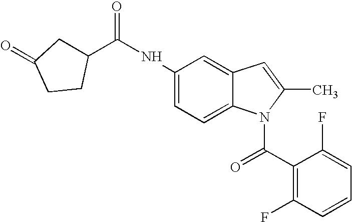 |
| 265 | 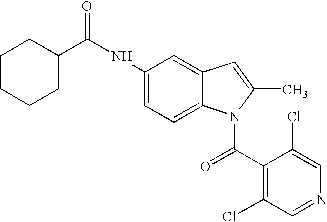 |
| 266 | 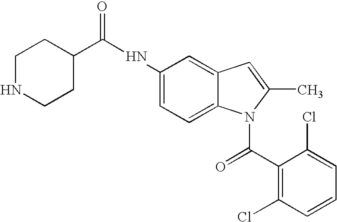 |
| 267 | 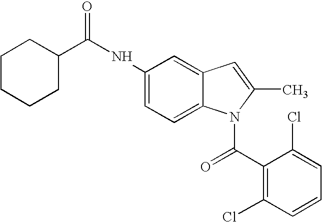 |
| 268 | 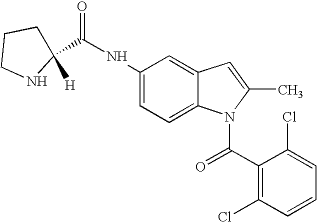 |
| 269 | 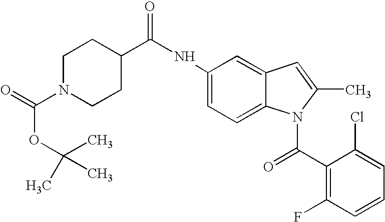 |
| 270 | 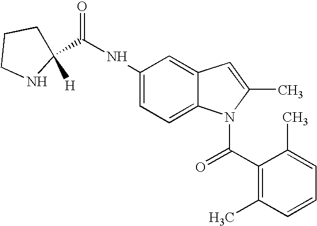 |
| 271 | 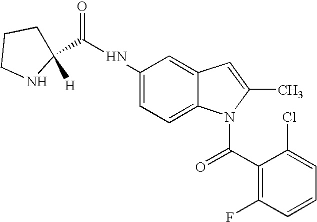 |
| 272 | 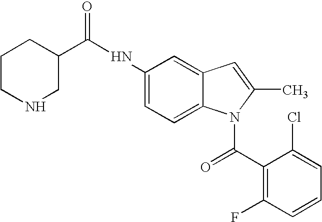 |
| 273 | 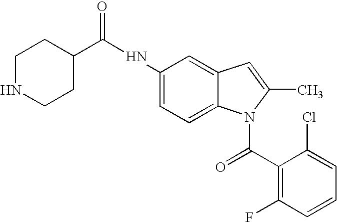 |

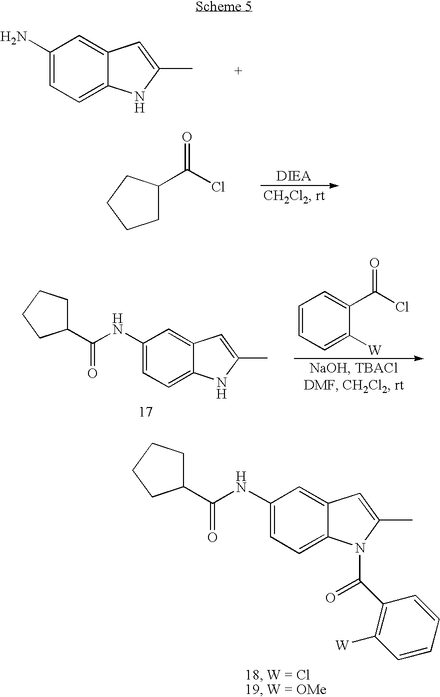

### 5-Cyclopentanecarbonylamino-2-methylindole (17)

Prepared as described in Procedure A above substituting 5-amino-2-methylindole for 5-amino-2-t-butylindole. ¹H NMR (CDCl₃) δ 8.11 (s, 1H), 7.66 (s, 2H), 7.34 (s, 1H), 7.13 (d, J=8.6 Hz, 1H), 6.11 (s, 1H), 2.68 (q, J=7.8 Hz, 1H), 2.38 (s, 3H), 1.93 (m, 4H), 1.81 (m, 2H), 1.62 (m, 2H). MS (m/z): 243 (MH⁺).

### 1-(2′-Chlorobenzoyl)-5-cyclopentanecarbonylamino-2-methylindole (18)

Prepared as described in Procedure B above. ¹H NMR (CDCl₃) δ 7.77 (d, J=2.1 Hz, 1H), 7.37 (m, 3H), 7.34 (m, 1H), 7.24 (s, 1H), 7.21 (s, 1H), 6.93 (dd, J=8.7, 2.1 Hz, 1H), 6.25 (s, 1H), 2.59 (q, J=8.1 Hz, 1H), 2.11 (s, 3H), 1.83 (m, 4H), 1.71 (m, 2H), 1.53 (m, 2H). MS (m/z): 381 (MH⁺).

### 1-(o-Anisoyl)-5-cyclopentanecarbonylamino-2-methylindole (19)

Prepared as described in Procedure B above. ¹H NMR (CDCl₃) δ 7.83 (d, J=1.5 Hz, 1H), 7.50 (t, J=7.8 Hz, 1H), 7.40 (dd, J=6.9, 1.2 Hz, 1H), 7.29 (d, J=9.0 Hz, 1H), 7.23 (s, 1H), 7.06 (t, J=7.2 Hz, 1H), 6.97 (d, J=8.4 Hz, 1H), 6.29 (s, 1H), 3.70 (s, 3H), 2.67 (q, J=8.1 Hz, 1H), 2.23 (s, 3H), 1.93 (m, 3H), 1.79 (m, 2H), 1.65 (m, 3H). MS (m/z): 377 (MH⁺).

The compounds shown in Table 5 were made in an analogous manner to that shown in Scheme 5, with obvious variation.

TABLE 5

| Com- | |
|---|---|
| pound | Structure |
| 274 | 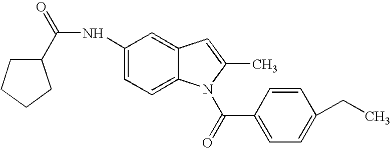 |
| 275 | 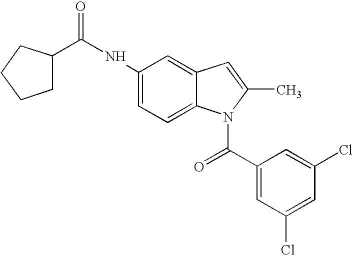 |
| 276 | 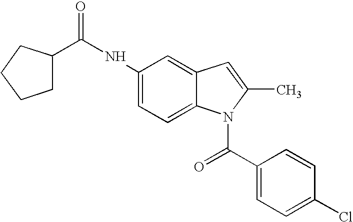 |
| 277 | 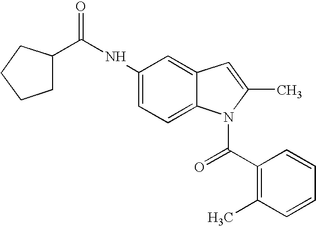 |

The compounds shown in Table 6 were purchased commercially from the indicated source.

TABLE 6

| Compound | Structure | Source | Catalog # |
|---|---|---|---|
| 278 | 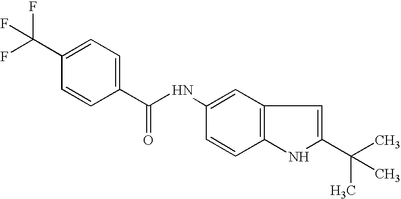 | Peakdale | PFC-1014 |
| 279 | 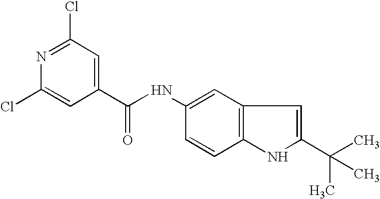 | Peakdale | PFC-1015 |
| 280 | 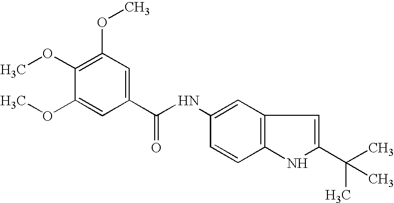 | Peakdale | PFC-1016 |
| 281 | 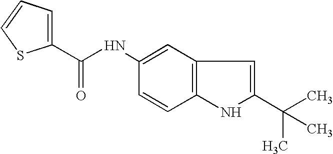 | Peakdale | PFC-1019 |
| 282 | 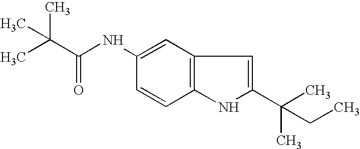 | Peakdale | PFC-2252 |
| 283 | 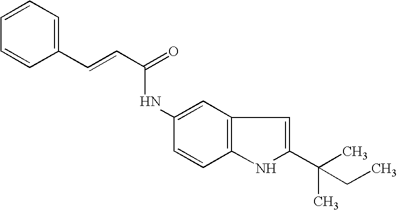 | Peakdale | PFC-2253 |
| 284 | 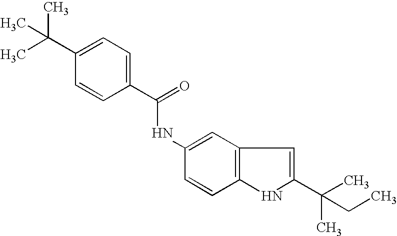 | Peakdale | PFC-2254 |
| 285 | 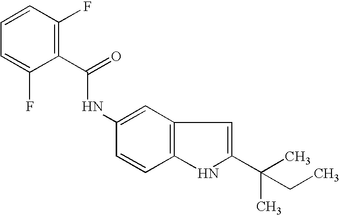 | Peakdale | PFC-2255 |
| 286 | 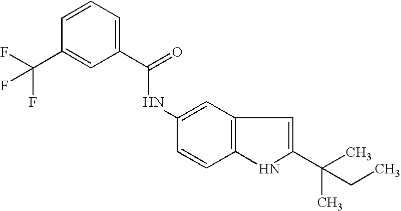 | Peakdale | PFC-2256 |
| 287 | 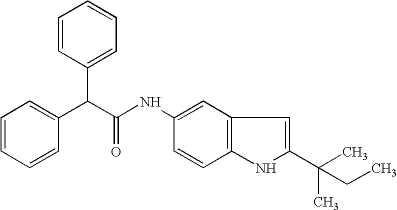 | Peakdale | PFC-2257 |
| 288 | 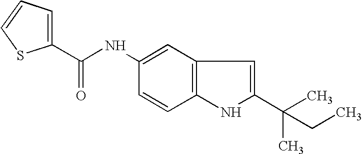 | Peakdale | PFC-2258 |
| 289 | 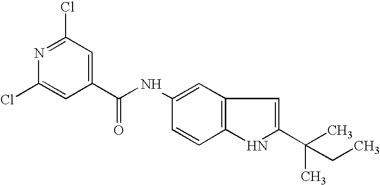 | Peakdale | PFC-2259 |
| 290 | 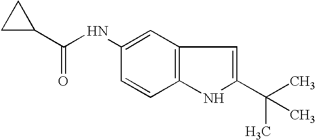 | Peakdale | PFC-1013 |

### BIOLOGY EXAMPLES

### Example 1

### HCV Replicon Assay

Actively dividing 5-2 Luc replicon cells were seeded at the density of 5000-7500 cells/well in the volume of 90 μl/well into 96 well plate(s). The cells were then incubated at 37° C. and 5% CO₂ for 24 hours. The 5-2 cells are replicon cells licensed from Ralf Bartenschlager (Germany) and have a self-replicating RNA molecule in the Huh7 cell; the RNA contains HCV non-structural proteins that make the self-replication possible.

Various concentrations of compounds (in the volume of 10 μl) were added into each well 24 hours after seeding the cells. The cells were incubated for another 24-48 hours before luciferase assay.

After incubating the 5-2 Luc replicon cells with the compounds for 24-48 hours, media were aspirated from each well and Bright-Glo (Promega) luciferase assay reagents were added to each well according to the manufacturer′s manual. Briefly, the Bright-Glo reagent was diluted with equal volume of PBS and an aliquote (100 μl) was added to each well. After incubating the plate at room temperature for 5 minutes, luciferase counts were taken using a luminometer.

### Example 2

### Immunoblotting Assay

Actively dividing 9-13 replicon cells (Huh7 cells comprising an HCV replicon) were seeded at the density of 1×10⁵ cells/well in the volume of 2 ml/well into 6 well plate(s). The cells were then incubated at 37° C. and 5% CO₂ for 24 hours.

Various concentrations of compounds (in the volume of 10 μl) were added into each well 24 hours after seeding the cells. The cells were incubated with the compounds for another 48 hours.

Protein samples were prepared from the cultured cells and resolved on a SDS-PAGE gel.

After electrophoresis, the protein samples on the SDS-PAGE gel were transferred to a nitrocellulose membrane.

The membrane was blocked with 5% non-fat milk in PBS for 1 hr at room temperature.

Primary antibody incubation was performed for 1 hour at room temperature before the membrane was washed for 3 times with PBST (PBS plus 0.1% Tween 20), 15 minutes each.

Horse Radish Peroxidase conjugated secondary antibody incubation was performed for 1 hour at room temperature before the membrane was washed for 3 times with PBST (PBS plus 0.1% Tween 20), 15 minutes each.

The membrane was then soaked in substrate solution (Pierce) and exposed to a film.

### Example 3

### TaqMan RT-PCR Assay

Actively dividing 9-13 replicon cells were seeded at the density of 3×10⁴ cells/well in the volume of 1 ml/well into 24 well plate(s). The cells were then incubated at 37° C. and 5% CO₂ for 24

Various concentrations of compounds (in the volume of 10 μl) were added into each well 24 hours after seeding the cells. The cells were incubated with the compounds for another 24-48 hours.

After incubating the 9-13 replicon cells with the compounds for 24-48 hours, media were aspirated off and RNA samples were prepared from each well.

TaqMan® (Roche Molecular Systems) one step RT-PCR was performed using the RNA samples according to the manufacturer's manual. Briefly, properly diluted RNA sample, upstream primer, downstream primer, FAM-labeled probe oligo were mixed and water was added to make up the volume to 25 μl. Equal volume of 2× TaqMan Master Mix were added and the reaction was performed in an ABI Prism 7700 Sequence Detector (Applied Biosystems).

The activity of a number of compounds according to the invention measured by the various assays is displayed in Table 7.

TABLE 7

| Compound | Assay | Activity |
|---|---|---|
| 279 | HCV Replicon Assay | a |
| 281 | HCV Replicon Assay | a |
| 281 | IMMUNOBLOTTING ASSAY | a |
| 283 | HCV Replicon Assay | b |
| 284 | HCV Replicon Assay | b |
| 285 | HCV Replicon Assay | a |
| 286 | HCV Replicon Assay | a |
| 287 | HCV Replicon Assay | b |
| 288 | HCV Replicon Assay | b |
| 289 | HCV Replicon Assay | a |
| 282 | HCV Replicon Assay | b |
| 290 | HCV Replicon Assay | a |
| 278 | HCV Replicon Assay | a |
| 280 | HCV Replicon Assay | b |
| 5a | HCV Replicon Assay | b |
| 5b | HCV Replicon Assay | b |
| 5c | HCV Replicon Assay | a |
| 5d | HCV Replicon Assay | b |
| 213 | HCV Replicon Assay | b |
| 214 | HCV Replicon Assay | b |
| 5e | HCV Replicon Assay | a |
| 215 | HCV Replicon Assay | b |
| 216 | HCV Replicon Assay | b |
| 217 | HCV Replicon Assay | b |
| 218 | HCV Replicon Assay | b |
| 219 | HCV Replicon Assay | b |
| 220 | HCV Replicon Assay | b |
| 221 | HCV Replicon Assay | b |
| 222 | HCV Replicon Assay | b |
| 223 | HCV Replicon Assay | a |
| 224 | HCV Replicon Assay | b |
| 225 | HCV Replicon Assay | b |
| 5f | HCV Replicon Assay | a |
| 226 | HCV Replicon Assay | b |
| 227 | HCV Replicon Assay | b |
| 5g | HCV Replicon Assay | a |
| 228 | HCV Replicon Assay | b |
| 229 | HCV Replicon Assay | b |
| 5h | HCV Replicon Assay | a |
| 9a | HCV Replicon Assay | a |
| 231 | HCV Replicon Assay | b |
| 232 | HCV Replicon Assay | b |
| 233 | HCV Replicon Assay | b |
| 234 | HCV Replicon Assay | b |
| 9b | HCV Replicon Assay | a |
| 235 | HCV Replicon Assay | b |
| 236 | HCV Replicon Assay | b |
| 9c | HCV Replicon Assay | a |
| 9d | HCV Replicon Assay | a |
| 9e | HCV Replicon Assay | a |
| 9f | HCV Replicon Assay | a |
| 13a | HCV Replicon Assay | a |
| 13b | HCV Replicon Assay | a |
| 13c | HCV Replicon Assay | a |
| 13d | HCV Replicon Assay | a |
| 13e | HCV Replicon Assay | a |
| 238 | HCV Replicon Assay | b |
| 237 | HCV Replicon Assay | b |
| 274 | HCV Replicon Assay | b |
| 239 | HCV Replicon Assay | b |
| 240 | HCV Replicon Assay | b |
| 241 | HCV Replicon Assay | b |
| 242 | HCV Replicon Assay | b |
| 243 | HCV Replicon Assay | b |
| 244 | HCV Replicon Assay | b |
| 16a-1 | HCV Replicon Assay | a |
| 16a-1 | RT-PCR Assay | a |
| 16a-12 | HCV Replicon Assay | a |
| 245 | HCV Replicon Assay | b |
| 246 | HCV Replicon Assay | b |
| 16a-13 | HCV Replicon Assay | a |
| 247 | HCV Replicon Assay | b |
| 248 | HCV Replicon Assay | b |
| 249 | HCV Replicon Assay | b |
| 250 | HCV Replicon Assay | b |
| 251 | HCV Replicon Assay | b |
| 252 | HCV Replicon Assay | b |
| 253 | HCV Replicon Assay | b |
| 254 | HCV Replicon Assay | b |
| 255 | HCV Replicon Assay | b |
| 16a-2 | HCV Replicon Assay | a |
| 16a-3 | HCV Replicon Assay | a |
| 16a-4 | HCV Replicon Assay | a |
| 16a-10 | HCV Replicon Assay | a |
| 16a-11 | HCV Replicon Assay | a |
| 256 | HCV Replicon Assay | b |
| 16b4 | HCV Replicon Assay | a |
| 16a-9 | HCV Replicon Assay | a |
| 16b-2 | HCV Replicon Assay | a |
| 16b-3 | HCV Replicon Assay | a |
| 16b-1 | HCV Replicon Assay | a |
| 16b-5 | HCV Replicon Assay | a |
| 16c-2 | HCV Replicon Assay | a |
| 16c-1 | HCV Replicon Assay | a |
| 16c-3 | HCV Replicon Assay | a |
| 16c-4 | HCV Replicon Assay | a |
| 16a-5 | HCV Replicon Assay | a |
| 16e-2 | HCV Replicon Assay | a |
| 16e-3 | HCV Replicon Assay | a |
| 16e-4 | HCV Replicon Assay | a |
| 16e-1 | HCV Replicon Assay | a |
| 257 | HCV Replicon Assay | b |
| 16e-5 | HCV Replicon Assay | a |
| 258 | HCV Replicon Assay | b |
| 259 | HCV Replicon Assay | b |
| 260 | HCV Replicon Assay | b |
| 260 | HCV Replicon Assay | b |
| 16a-6 | HCV Replicon Assay | a |
| 16a-7 | HCV Replicon Assay | a |
| 16a-8 | HCV Replicon Assay | a |
| 16b-6 | HCV Replicon Assay | a |
| 16a-14 | HCV Replicon Assay | a |
| 16c-5 | HCV Replicon Assay | a |
| 262 | HCV Replicon Assay | b |
| 16d-3 | HCV Replicon Assay | b |
| 16d-1 | HCV Replicon Assay | b |
| 16d-2 | HCV Replicon Assay | b |
| 263 | HCV Replicon Assay | b |
| 264 | N/D | |
| 18 | HCV Replicon Assay | b |
| 275 | HCV Replicon Assay | b |
| 276 | HCV Replicon Assay | b |
| 19 | HCV Replicon Assay | a |
| 277 | HCV Replicon Assay | b |
| 230 | HCV Replicon Assay | b |

| | | |
|---|---|---|
| 16g-1 | HCV Replicon Assay | a |
| 16g-2 | HCV Replicon Assay | a |
| 265 | HCV Replicon Assay | b |
| 16g-3 | HCV Replicon Assay | a |
| 16f-5 | HCV Replicon Assay | a |
| 16f-2 | HCV Replicon Assay | a |
| 16f-3 | HCV Replicon Assay | a |
| 16f-4 | HCV Replicon Assay | a |
| 16f-1 | HCV Replicon Assay | a |
| 266 | HCV Replicon Assay | a |
| 267 | HCV Replicon Assay | b |
| 268 | HCV Replicon Assay | b |
| 269 | HCV Replicon Assay | b |
| 270 | HCV Replicon Assay | b |
| 271 | HCV Replicon Assay | b |
| 272 | HCV Replicon Assay | b |
| 273 | HCV Replicon Assay | b |

“a” indicates inhibitory activity at a concentration of less than 10 micromolar;
“b” indicates activity is at a concentration greater than 10 micromolar.

1. A compound of formula (I): 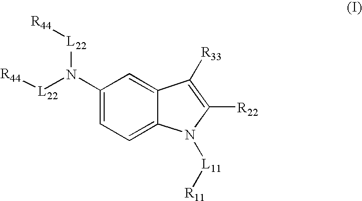
or a pharmaceutically acceptable salt thereof, wherein:
L¹¹ is carboxyl, or a covalent bond when R¹¹ is H;
R¹¹ is H except when L¹¹ is carboxyl, phenyl substituted with 1-3 R⁵⁰, or C₄₋₆-heteroaryl containing 1-3 heteroatoms selected from the group N, S, and O and substituted with 1-3 R⁵⁰;
R²² is H, or C₁₋₆ alkyl, such as CH₃, t-butyl, or neo-pentyl;
R³³ is H, CH₃ or C₁₋₃ alkyl;
each L²² is independently carboxyl (C(O)), C₁₋₄ alkyl, C₁₋₄ alkylC(O) or a covalent bond;
each R⁴⁴ is independently H, optionally substituted C₁₋₆ alkyl, optionally substituted C₃₋₇ cycloalkyl, optionally substituted C₃₋₇ heterocycloalkyl containing at least one N, O or S atom,
C₃₋₇ cycloalkanone, optionally substituted C₃₋₇ monocyclic or C₇₋₁₃ bicyclic aryl, optionally substituted C₃₋₆ monocyclic or C₅₋₁₃ bicyclic heteroaryl containing at least one N, O, or S atom, or optionally substituted C₃₋₆ monocyclic or C₅₋₁₃ bicyclic heterocycle containing at least one N, O, or S atom, wherein said optional substitutions are one to four R⁶ groups;
each R⁵⁰ is independently H, halo, Cl, F, CF₃, C₁-C₃ per fluoro, C₁-C₃ perhalo, —OC₁-C₃ perhalo, NO₂, CH₃, R⁷, —OCH₃, —OR⁷, —SR⁷, —CN, —NHR⁷, —N(R⁷)₂, —CON(H)R²³CON(R⁷)₂|, —R²³ N(H)R^{7|}, —R²³ N(R⁷)₂;
each R⁶ is independently H, halo, Cl, F, —CF₃, —NO₂, —R⁵⁰, —SR⁵⁰, —OR⁵⁰, —CN, N(R⁵⁰)₂, —C(O)R⁵⁰, —R²³C(O)R⁵⁰, —CON(R⁵⁰)₂, C₄-C₆ cycloalkyl, C₃₋₇ cycloalkanone, C₄₋₆ cycloalkylamine, C₃₋₆ monocyclic or C₅₋₁₃ bicyclic heteroaryl containing at least one N, O, or S atoms or a C₆-C₁₂ monocyclic or bicyclic heterocycle containing at least one N, O, or 5 atom;
R⁷ is H, halo or C₁₋₆ alkyl;
R²³ is a bond or is C₁-C₆ alkyl;
with the proviso that R²² is not CH₃ when R¹¹ is H;
with the further proviso that the compound is not:

| | |
|---|---|
| 278 | 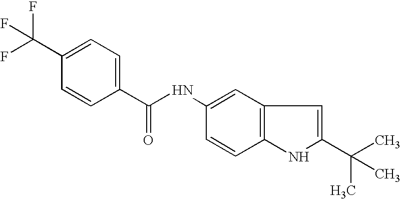 |
| 279 | 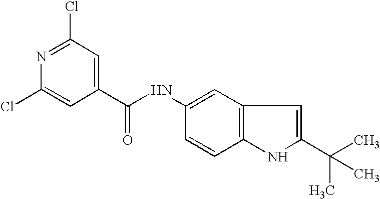 |
| 280 | 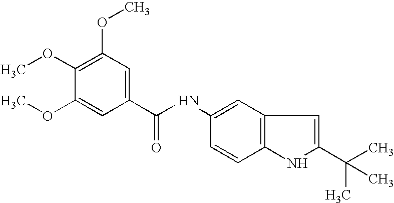 |
| 281 | 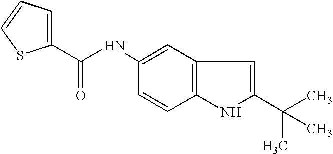 |
| 282 | 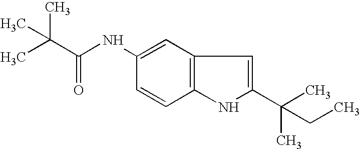 |
| 283 | 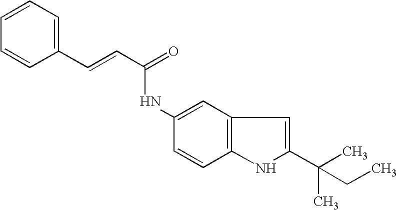 |
| 284 | 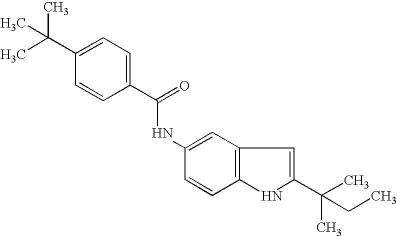 |
| 285 | 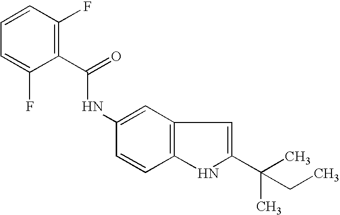 |
| 286 | 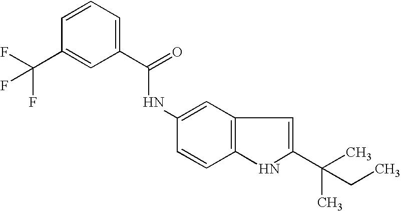 |
| 287 | 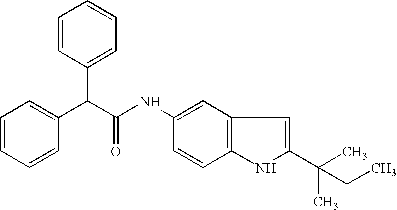 |
| 288 | 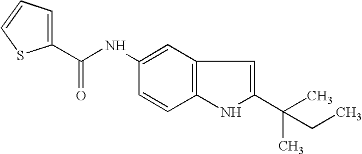 |
| 289 | 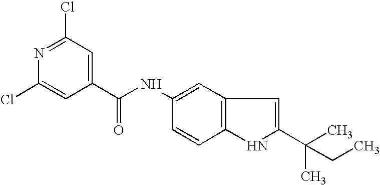 |
| 290 | 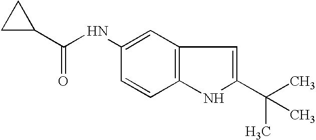 |
| 278 | 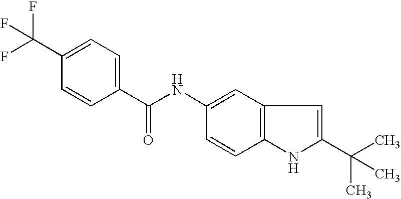 |
| 280 | 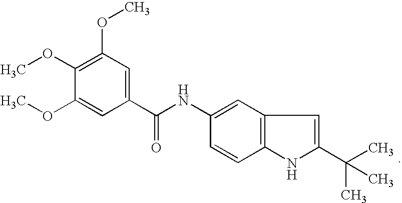 |

2. A compound according to claim 1 wherein at least one L²² is carboxyl.

3. A compound according to claim 2 wherein the R⁴⁴ attached to said at least one L²² carboxyl is optionally substituted C₁₋₆ alkyl, optionally substituted C₃₋₇ cycloalkyl, optionally substituted C₃₋₇ heterocycloalkyl containing at least one N, O or S atom, C₃₋₇ cycloalkanone, optionally substituted C₅₋₇ monocyclic or C₃₋₁₃ bicyclic aryl, optionally substituted C₃₋₆ monocyclic or C₅₋₁₃ bicyclic heteroaryl containing at least one N, O, or S atom, or optionally substituted C₃-C₁₃ monocyclic or bicyclic heterocycle containing at least one N, O, or S atom.

4. A compound according to claim 4 wherein R⁴⁴ is optionally substituted C₃₋₇ cycloalkyl, optionally substituted C₃₋₇ heterocycloalkyl containing at least one N, O or S atom, optionally substituted C₅₋₇ monocyclic aryl, or optionally substituted C₃₋₆ monocyclic heteroaryl containing at least one N, O, or S.

5. A compound according to claim 3 wherein R⁴⁴ is optionally substituted C₃₋₇ cycloalkyl, optionally substituted C₅₋₇ monocyclic aryl, or optionally substituted C₃₋₆ monocyclic heteroaryl containing at least one N, O, or S.

6. A compound according to claim 1 wherein R²² is t-butyl or Neopentyl.wherein R¹¹ is H.

7. A compound according to claim 6 wherein R³³ is H.

8. A compound according to claim 5 wherein R²² is t-butyl.

9. A compound according to claim 8 wherein R³³ is H.

10. A compound according to claim 1 wherein said compound is selected from the group consisting of:

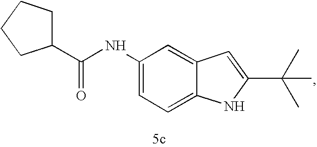

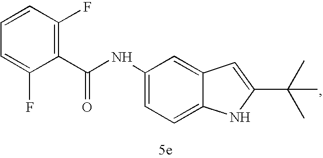

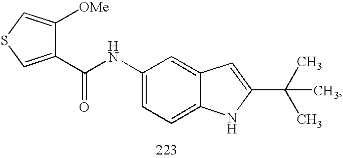

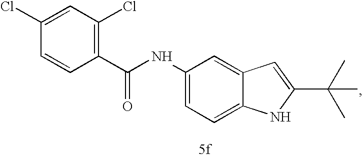

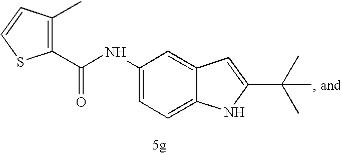

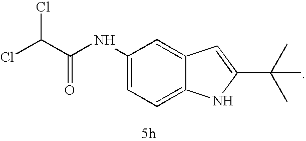

11. A compound according to claim 5 wherein R²² is neo-pentyl.

12. A compound according to claim 11 wherein R³³ is H.

13. A compound according to claim 1 wherein L¹¹ is carboxyl.

14. A compound according to claim 13 wherein R¹¹ is Phenyl or Pyridyl.

15. A compound according to claim 3 wherein L¹¹ is carboxyl.

16. A compound according to claim 15 wherein R¹¹ is Phenyl or Pyridyl.

17. A compound according to claim 16 wherein R²² is CH₃.

18. A compound according to claim 17 wherein R³³ is H or CH₃.

19. A compound according to claim 16 wherein R¹¹ is substituted with one or two substituents independently selected from halo, Cl, F, CF₃, CH₃ or —OCH₃.

20. A compound according to claim 19 wherein R²² is CH₃.

21. A compound according to claim 19 wherein R³³ is H or CH₃.

22. A compound according to claim 20 wherein R³³ is H or CH₃.

23. A compound according to claim 1 selected from the group consisting of:

| | |
|---|---|
| 5-(Cyclopentanecarbonyl)amino-1-(2′,6′- dichlorobenzoyl)-3-methylindole, | 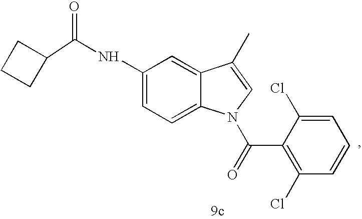 |
| 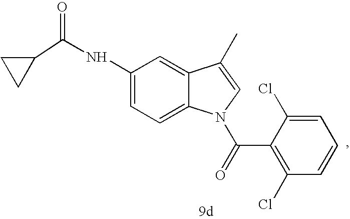 | 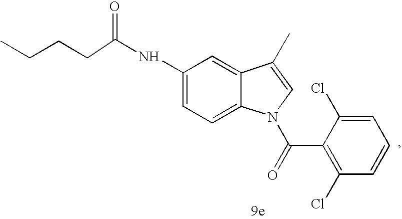 |
| 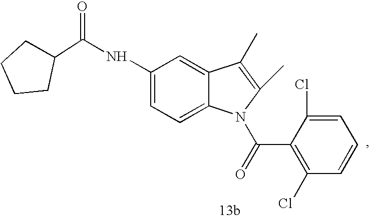 | 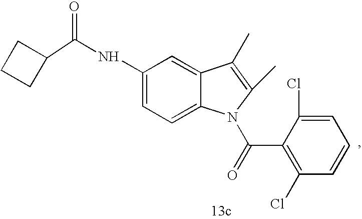 |
| 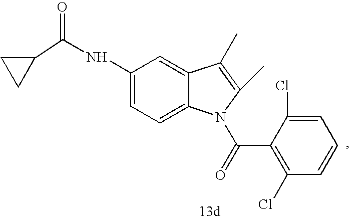 | 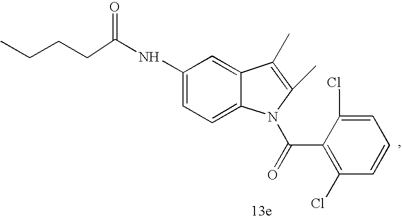 |
| 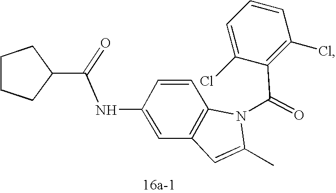 | 1-(2′,6′-Dichlorobenzoyl)-5-(4-methoxy-3- thiophenylcarbonyl)amino-2-methylindole, |
| 1-(2′,6′-Dichlorobenzoyl)-5-(3-pyridyl-2- | 5-Cyclohexanecarbonylamino-1-(2′,6′- |
| acetamido)-2-methylindole, | dichlorobenzoyl)-2-methylindole, |
| 5-Cyclobutanecarbonylamino-1-(2′,6′- | 1-(2′,6′-Dichlorobenzoyl)-5-(3-methyl-2- |
| dichlorobenzoyl)-2-methylindole, | thiophenylcarbonyl)amino-2-methylindole, |
| 1-(2′,6′-Dichlorobenzoyl)-5-(2- | 1-(2′,6′-Dichlorobenzoyl)-5-(2- |
| ethylbutanoyl)amino-2-methylindole, | methylpropanoyl)amino-2-methylindole, |
| 1-(2′-Chloro-6′-fluorobenzoyl)-5- | 1-(2′-Chloro-6′-fluorobenzoyl)-5- |
| Cyclohexanecarbonylamino-2-methylindole, | cyclobutanecarbonylamino-2-methylindole, |
| 1-(2′-Chloro-6′-fluorobenzoyl)-5- | 1-(2′-Chloro-6′-fluorobenzoyl)-5- |
| cyclopropanecarbonylamino-2-methylindole, | cyclopentanecarbonylamino-2-methylindoie, |
| 1-(2′-Chloro-6-fluorobenzoyl)-5-(3-oxo-1- | 1-(2′,6′-Dichlorobenzoyl)-5-(2- |
| cyclopentanecarbonyl)amino-2-methylindole, | methylbutanoyl)amino-2-methylindole, |
| 1-(2′,6′-Dichlorobenzoyl)-5-(n-pentanoyl)amino- | 1-(2′,6′-Dimethoxybenzoyl)-5-(3-oxo-1- |
| 2-methylindole, | cyclopentanecarbonyl)amino-2-methylindole, |
| 1-(2′,6′-Dichlorobenzoyl)-5-(3-oxo-1- | 1-(2′-Fluoro-6′-trifluoromethylbenzoyl)-5-(3-oxo- |
| cyclopentanecarbonyl)amino-2-methylindole, | 1-cyclopentanecarbonyl)amino-2- |
| | methylindole, |
| 5-Cyclopropanecarbonylamino-1-(2′,6′- | 5-Cyclopentanecarbonylamino-1-(2′,6′- |
| difluorobenzoyl)-2-methylindole, | difluorobenzoyl)-2-methylindole, |
| 5-Cyclobutanecarbonylamino-1-(2′,6′- difluorobenzoyl)-2-methylindole, | 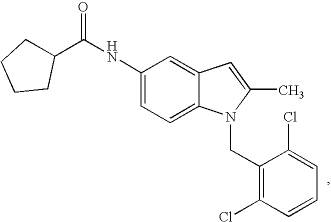 |
| 1-(2′-Chlorobenzoyl)-5- | 1-(o-Anisoyl)-5-cyclopentanecarbonylamino-2- |
| cyclopentanecarbonylamino-2-methylindole, | methylindole, |
| 5-Cyclopentanecarbonylamino-1-(2′,6′- | |
| dichloro-4′-pyridylcarbonyl)-2-methylindole, | |
| 1-(2′,6′-Dichloro-4-pyridylcarbonyl)-5-(3-oxo-1- | 5-Cyclohexanecarbonylamino-1-(2′,6′- |
| cyclopentanecarbonyl)amino-2-methylindole, and | dimethylbenzoyl)-2-methylindole. |

24. A compound according to claim 1 wherein at least one L²² is a bond and the R⁴⁴ attached thereto is H.

25. A pharmaceutical composition comprising a compound according to claim 1 and a pharmaceutically acceptable carrier, excipient, or diluent.

26. A pharmaceutical composition according to claim 25 wherein for said compound at least one L²² is carboxyl.

27. A pharmaceutical composition according to claim 26 wherein for said compound the R⁴⁴ attached to said at least one L²² carboxyl is optionally substituted C₁₋₆ alkyl, optionally substituted C₃₋₇ cycloalkyl, optionally substituted C₃₋₇ heterocycloalkyl containing at least one N, O or S atom, C₃₋₇ cycloalkanone, optionally substituted C₅₋₇ monocyclic or C₃₋₁₃ bicyclic aryl, optionally substituted C₃₋₆ monocyclic or C₅₋₁₃ bicyclic heteroaryl containing at least one N, O, or S atom, or optionally substituted C₃-C₁₃ monocyclic or bicyclic heterocycle containing at least one N, O, or S atom.

28. A pharmaceutical composition according to claim 27 wherein -for said compound R²² is t-butyl or neo-pentyl.

29. A pharmaceutical composition according to claim 28 wherein said compound is selected from the group consisting of:

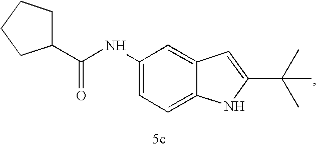

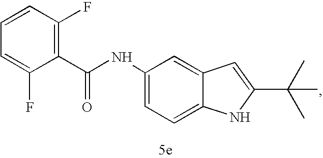

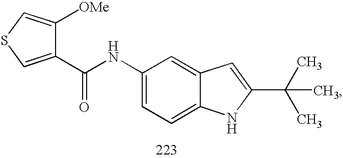

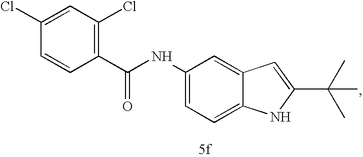

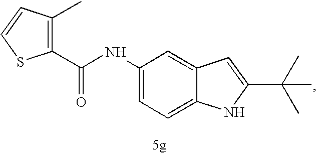

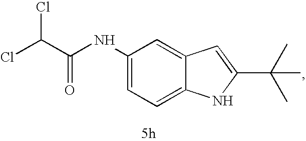

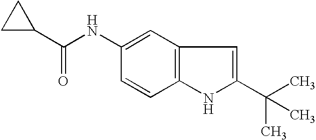

| | |
|---|---|
| 278 | 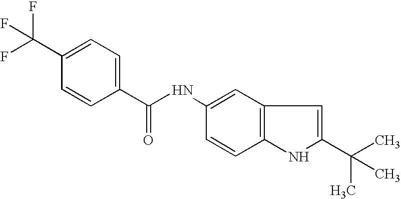 |
| 279 | 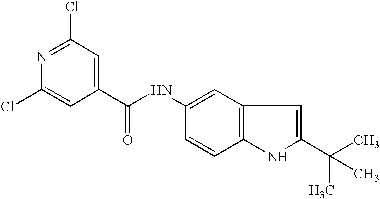 |
| 280 | 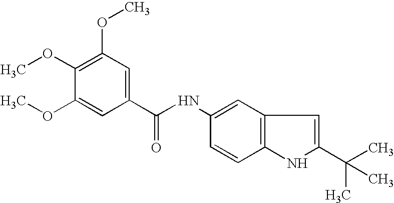 |
| 281 | 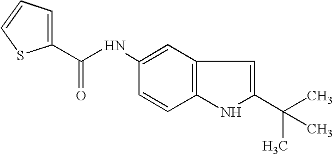 |
| 282 | 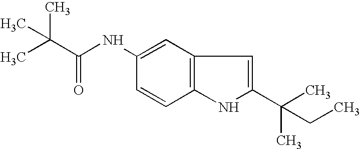 |
| 283 | 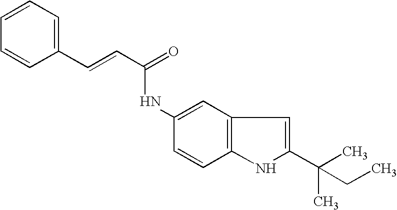 |
| 284 | 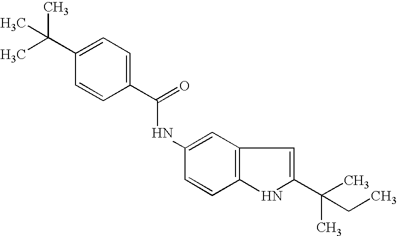 |
| 285 | 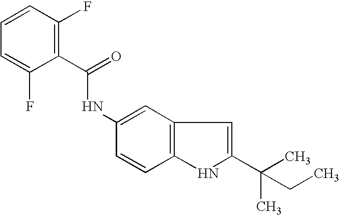 |
| 286 | 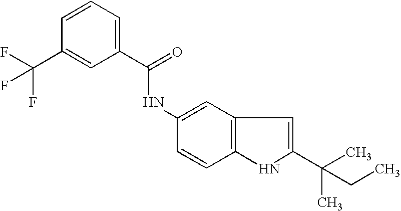 |
| 287 | 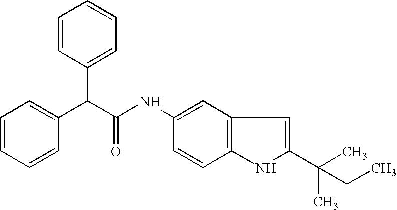 |
| 288 | 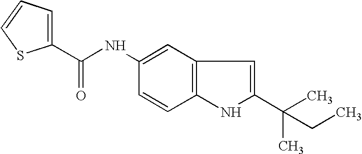 |
| 289 | 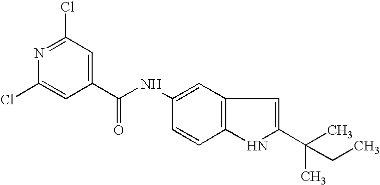 |
| 290 | 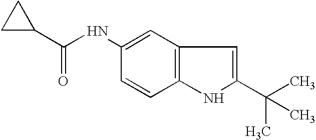 |
| 278 | 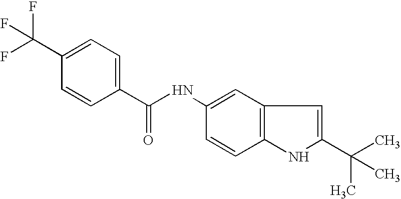 |
| 280 | 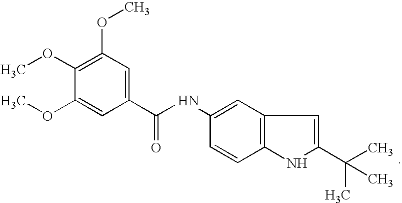 |

30. A pharmaceutical composition according to claim 27 wherein for said compound L¹¹ is carboxyl.

31. A pharmaceutical composition according to claim 30 wherein for said compound R¹¹ is phenyl or pyridyl.

32. A pharmaceutical composition according to claim 31 wherein for said compound R²² is CH₃.

33. A pharmaceutical composition according to claim 32 wherein for said compound R³³ is H or CH₃.

34. A pharmaceutical composition according to claim 31 wherein for said compound R³³ is H or CH₃.

35. A pharmaceutical composition according to claim 25 wherein for said compound at least one L²² is a bond and the R⁴⁴ attached thereto is H.

36. A method of inhibiting hepatitis C virus (HCV) proliferation comprising contacting and HCV infected cell with a compound according to claim 1.

37. A method of inhibiting hepatitis C virus (HCV) proliferation comprising contacting and HCV infected cell with a compound according to claim 24.

38. A method of treating a mammal infected with HCV, said method comprising administering to said mammal a therapeutically effective amount of a composition according to claim 25.

39. A method of treating a mammal infected with HCV, said method comprising administering to said mammal a therapeutically effective amount of a composition according to claim 35.

40. The method of claim 38, wherein said mammal is a human.

41. The method of claim 39, wherein said mammal is a human.
